SEQUENCE LISTING (57 sequences)

SEQ ID NO: 1 - protein, 8 aa, Homo sapiens
GFDFSRNG

SEQ ID NO: 2 - protein, 7 aa, Homo sapiens
VTFESKT

SEQ ID NO: 3 - protein, 21 aa, Homo sapiens
VKDQTIFHKNGAVDFFSYFDL

SEQ ID NO: 4 - protein, 12 aa, Homo sapiens
HSLLYAPYDKDA

SEQ ID NO: 5 - protein, 3 aa, Homo sapiens
WAS

SEQ ID NO: 6 - protein, 9 aa, Homo sapiens
QQTRWTPPT

SEQ ID NO: 7 - DNA, 24 bp, Homo sapiens
ggattcgacttcagtcgcaatggg

SEQ ID NO: 8 - DNA, 21 bp, Homo sapiens
gtcaccttcgaaagtaagaca

SEQ ID NO: 9 - DNA, 63 bp, Homo sapiens
gttaaagaccagactatttttcacaaaaatggagccgtcgatttcttctcgtacttcgacctg

SEQ ID NO: 10 - DNA, 36 bp, Homo sapiens
cacagtcttttatatgccccctacgataaggatgcc

SEQ ID NO: 11 - DNA, 9 bp, Homo sapiens
tgggcatca

SEQ ID NO: 12 - DNA, 27 bp, Homo sapiens
caacagacccgctggactccccctacc

SEQ ID NO: 13 - protein, 127 aa, Homo sapiens
QMKLMQSGGVMVRPGESATLSCVASGFDFSRNGFEWLRQGPGKGLQWLATVTFESKTHVTASARGRFTISRDNSRRTVYLQMTNLQPDDTAMYFCVKDQTIFHKNGAVDFFSYFDLWGRGAPVIVSA

SEQ ID NO: 14 - protein, 114 aa, Homo sapiens
DVVMTQSPEFLAVSLGERATLECKSSHSLLYAPYDKDALVWYQQKPGQPPKLLLDWASSRRSGVSDRFSATSASGRYFTLTISNFRADDVATYYCQQTRWTPPTFGGGTKVDLN

SEQ ID NO: 15 - DNA, 382 bp, Homo sapiens
cagatgaagttgatgcagtcggggggagtcatggtccggcctggagaatcggcgacactgtcttgtgttgcctctggattcgacttcagtcgcaatgggttcgagtggctccgccagggtcccggcaaggggctgcagtggctggccacagtcaccttcgaaagtaagacacatgtcacggcctccgcgcggggccgattcactatttctagagacaattccaggagaaccgtctatttgcaaatgactaatctgcagcctgacgatacggcaatgtatttctgtgttaaagaccagactatttttcacaaaaatggagccgtcgatttcttctcgtacttcgacctgtggggccgtggcgccccggtcatagtctccgcag

SEQ ID NO: 16 - DNA, 343 bp, Homo sapiens
gacgtcgtgatgacccagtctccagaattcctggctgtgtctctgggcgagagggccaccctcgaatgcaagtccagccacagtcttttatatgccccctacgataaggatgccttagtttggtatcagcaaaaaccagggcagcctccaaagttgctccttgactgggcatcaagtcgtcgctccggggtctctgaccgattcagtgccaccagcgcatctgggagatactttactcttacgattagcaatttccgggctgacgacgtggcgacttattattgtcaacagacccgctggactccccctaccttcggcggcgggaccaaggtggatctcaacc

SEQ ID NO: 17 - protein, 8 aa, Homo sapiens
GGTFNRLA

SEQ ID NO: 18 - protein, 8 aa, Homo sapiens
IMPIFDIT

SEQ ID NO: 19 - protein, 15 aa, Homo sapiens
ARASYSSSSPYAFDI

SEQ ID NO: 20 - protein, 6 aa, Homo sapiens
QDIRNH

SEQ ID NO: 21 - protein, 3 aa, Homo sapiens
DAS

SEQ ID NO: 22 - protein, 10 aa, Homo sapiens
QHYDDLPRIT

SEQ ID NO: 23 - DNA, 24 bp, Homo sapiens
ggaggcaccttcaacagattagct

SEQ ID NO: 24 - DNA, 24 bp, Homo sapiens
atcatgcctatatttgatataaca

SEQ ID NO: 25 - DNA, 45 bp, Homo sapiens
gcgagagcatcctatagcagctcgtccccttatgcttttgatatc

SEQ ID NO: 26 - DNA, 18 bp, Homo sapiens
caagacattaggaaccat

SEQ ID NO: 27 - DNA, 9 bp, Homo sapiens
gatgcatcc

SEQ ID NO: 28 - DNA, 30 bp, Homo sapiens
caacactatgatgatctgcctcgaatcacc

SEQ ID NO: 29 - protein, 122 aa, Homo sapiens
QVQLVQSGAEVKKPGSSVKVSCKTSGGTFNRLAMSWVRQAPGQGLEWMGGIMPIFDITNYAQKFQGRVTIITDESTSTAYMELRSLTSEDSAVYYCARASYSSSSPYAFDIWGQGTMVTVSS

SEQ ID NO: 30 - protein, 108 aa, Homo sapiens
DIQMTQSPSSLSPSVGDRVTITCQASQDIRNHLNWYQQKPGKAPKLLIYDASNLATGVPSRFSGSGSGTDFTFTISSLQPEDLATYYCQHYDDLPRITFGQGTRLEIK

SEQ ID NO: 31 - DNA, 367 bp, Homo sapiens
caggtccagttggtgcagtctggggctgaggtgaagaagcctgggtcctcggtgaaggtctcctgcaagacctctggaggcaccttcaacagattagctatgagctgggtgcgacaggcccctggacaagggcttgagtggatgggagggatcatgcctatatttgatataacaaactatgcacagaagttccagggcagagtcacgattatcacggacgaatccacgagcacagcctacatggaactgaggagcctgacatctgaggactcggccgtgtattattgtgcgagagcatcctatagcagctcgtccccttatgcttttgatatctggggccaagggacaatggtcaccgtctcttcag

SEQ ID NO: 32 - DNA, 325 bp, Homo sapiens
gacatccagatgacccagtctccatcctccctgtctccatctgtaggagacagagtcaccatcacttgccaggcgagtcaagacattaggaaccatttaaattggtatcagcagaaaccagggaaagcccctaaactcctgatctacgatgcatccaatttggcaacaggggtcccatcaaggttcagtggaagtggatctgggacagattttactttcaccatcagcagcctgcagcctgaagatcttgcaacatattactgtcaacactatgatgatctgcctcgaatcaccttcggccaagggacacgactggagattaaac

SEQ ID NO: 33 - protein, 8 aa, Homo sapiens
GYNFNDYW

SEQ ID NO: 34 - protein, 8 aa, Homo sapiens
IYPDDSDT

SEQ ID NO: 35 - protein, 12 aa, Homo sapiens
ARLDGDYADFDS

SEQ ID NO: 36 - protein, 8 aa, Homo sapiens
NSNLGDKF

SEQ ID NO: 37 - protein, 3 aa, Homo sapiens
DND

SEQ ID NO: 38 - protein, 10 aa, Homo sapiens
GTWDNSLSAL

SEQ ID NO: 39 - DNA, 24 bp, Homo sapiens
ggatacaactttaacgactactgg

SEQ ID NO: 40 - DNA, 24 bp, Homo sapiens
atctatcctgatgactccgatact

SEQ ID NO: 41 - DNA, 36 bp, Homo sapiens
gcgagactcgacggtgactacgccgattttgactcc

SEQ ID NO: 42 - DNA, 24 bp, Homo sapiens
aactccaaccttggcgataagttt

SEQ ID NO: 43 - DNA, 9 bp, Homo sapiens
gacaatgat

SEQ ID NO: 44 - DNA, 30 bp, Homo sapiens
ggaacatgggataacagcctgagtgcctta

SEQ ID NO: 45 - protein, 119 aa, Homo sapiens
EVQLVQSGAEVKKPGESLKISCYGSGYNFNDYWIGWVRQMPGKGLEWMGMIYPDDSDTKYSAAFEGQVTLSADKSISTAYLQWTSLRASDTATYYCARLDGDYADFDSWGQGTLVTVSS

SEQ ID NO: 46 - protein, 109 aa, Homo sapiens
QSVLTQPPSVSAAPGQKVTISCSGTNSNLGDKFVSWYQQLPGTAPRLLIYDNDKRPSGIPDRFSGSKSGASATLAITGLQTGDEADYYCGTWDNSLSALFGGGTKLTVL

SEQ ID NO: 47 - DNA, 358 bp, Homo sapiens
gaggtgcagctggtgcagtctggagcagaggtgaaaaagcccggagagtctctgaagatctcctgttacggctctggatacaactttaacgactactggatcggctgggtgcgccagatgcccgggaaaggcctggagtggatgggtatgatctatcctgatgactccgatactaaatacagtgcggccttcgaaggccaggtcaccctgtcagccgacaagtccatcagcaccgcctacctgcaatggaccagcctgcgggcctcggacaccgccacgtattactgtgcgagactcgacggtgactacgccgattttgactcctggggccagggaaccctggtcaccgtctcctcac

SEQ ID NO: 48 - DNA, 328 bp, Homo sapiens
cagtctgtgttgacgcagccgccctcagtgtctgcggcccccggacagaaggtcaccatctcctgctctggaaccaactccaaccttggcgataagtttgtatcctggtaccaacaactcccaggaacagcccccagactcctcatttatgacaatgataagcgaccctcaggcattcctgaccgattctctggctccaagtctggcgcgtcagccaccctggccatcaccggactccagactggggacgaggccgattattactgcggaacatgggataacagcctgagtgccttattcggcggagggaccaagctgaccgtcctac

SEQ ID NO: 49 - protein, 12 aa, Homo sapiens
RRSVRIGPGQTF

SEQ ID NO: 50 - protein, 4 aa, Homo sapiens
GPGR

SEQ ID NO: 51 - protein, 4 aa, Homo sapiens
GPGQ

SEQ ID NO: 52 - protein, 4 aa, Homo sapiens
GPGK

SEQ ID NO: 53 - protein, 4 aa, Homo sapiens
APGR

SEQ ID NO: 54 - protein, 12 aa, Homo sapiens
RKSVRIGPGQTF

SEQ ID NO: 55 - protein, 15 aa, Homo sapiens
QQHLLQLTVWGIKQL

SEQ ID NO: 56 - protein, 13 aa, Homo sapiens
TVYALFYRLDIVP

SEQ ID NO: 57 - protein, 13 aa, Homo sapiens
TNLIYTLIEESQN

(19) United States
(12) Patent Application Publication
(10) Pub. No.: US 2011/0212106 A1
(43) Pub. Date: Sep. 1, 2011

(54) HIV-1 NEUTRALIZING ANTIBODIES AND USES THEREOF

(75) Inventors: Antonio Lanzavecchia, Bellinzona (CH); Davide Corti, Bellinzona (CH)

(73) Assignee: INSTITUTE FOR RESEARCH IN BIOSCIENCE, Bellinzona (CH)

(21) Appl. No.: 13/010,336

(22) Filed: Jan. 20, 2011

Related U.S. Application Data
(60) Provisional application No. 61/296,636, filed on Jan. 20, 2010.

(51) Int. Cl. A61K 39/42; C12N 5/10; C07K 16/10; C07H 21/00; C12N 1/00; A61K 31/7088; A61K 48/00; A61K 39/21; C12Q 1/70; C12N 15/63; C07K 2/00; G01N 33/15; A61P 37/04; A61P 31/18

(52) U.S. Cl. 424/160.1; 435/339.1; 530/389.4; 530/387.3; 530/387.9; 536/23.53; 435/243; 435/419; 514/44 R; 424/93.2; 424/208.1; 435/5; 435/320.1; 530/350; 436/501

(57) ABSTRACT

The invention relates to antibodies and antigen binding fragments thereof that neutralize HIV-1 infection. The invention also relates to nucleic acids that encode, immortalized B cells and cultured plasma cells that produce, and to epitopes that bind to such antibodies and antibody fragments. In addition, the invention relates to the use of the antibodies, antibody fragments, and epitopes in screening methods as well as in the diagnosis, treatment and prevention of HIV-1 infection.

This application claims the benefit of priority of U.S. provisional Application No. 61/296,636, filed Jan. 20, 2010, the disclosures of which is hereby incorporated by reference as if written herein in its entirety.

### BACKGROUND

Human immunodeficiency virus type 1 (HIV-1) is characterized by an extraordinary genetic diversity, reflected by the presence of several clades (subtypes), a fact that represents a significant impediment to vaccine development. Env is the most variable HIV-1 gene, with up to 35% sequence diversity among clades, 20% diversity within clades, and 10% diversity in a single infected individual. Neutralizing antibodies provide one arm of the adaptive immune response against HIV-1. HIV-1 neutralizing monoclonal antibodies (mAbs) protect against vaginal or intravenous challenge with SIV-HIV-1 chimeric viruses (SHIV). Protection may depend not only on viral neutralization but also on Fc-mediated antibody effector functions. Given the predicted low-titer inoculum driving HIV-1 sexual transmission, a vaccine capable of eliciting antibodies that neutralize a broad spectrum of viral strains could potentially reduce or prevent infection.

Several studies have questioned the existence of broadly neutralizing antibodies as part of a normal immune response to HIV-1 infection. Indeed, the HIV-1-neutralizing mAb b12 isolated from a phage library, and the mAbs 2F5, 4E10 and 2G12, although isolated from memory B cells, do not appear to have a counterpart in human sera. This notion is also supported by a recent study by Nussenzweig and co-workers who used a trimeric gp140 to isolate antigen-binding memory B cells from which 500 antibody sequences were retrieved by single cell PCR (see Scheid, J. F., et al. (2009) Nature 458, 636-640). Although the donors had broadly neutralizing serum activity, none of the mAbs isolated had such a property, raising the possibility that the broad serum neutralizing activity results from multiple clonal responses against different epitopes. Broadly neutralizing mAbs can be used to define epitopes and to facilitate the template-based design of immunogens for the development of a vaccine able to induce neutralizing antibodies against the wide range of HIV-1 strains present in the global pandemic.

Identification of broadly neutralizing mAbs from HIV-1 infected individuals, and the characterization of their cognate epitopes will be instrumental in the design of immunogens capable of eliciting such a broad neutralizing response. Accordingly, there is a need for human mAbs that neutralize a broad spectrum of primary HIV-1 isolates and the characterization of the human neutralizing antibody B cell response to HIV-1 infection are important goals that are central to the design of an effective antibody-based vaccine.

### SUMMARY

The invention is based, in part, on the discovery of broadly neutralizing antibodies that neutralize HIV-1. Accordingly, in one aspect of the invention, the invention comprises a human antibody, an antibody variant, or an antigen binding fragment thereof, that neutralizes HIV-1.

In yet another embodiment, the invention comprises an antibody, or an antigen binding fragment thereof, comprising at least one complementarity determining region (CDR) sequence having at least 95% sequence identity to any one of SEQ ID NOs: 1-6, 17-22, or 33-38, wherein the antibody neutralizes HIV-1 infection.

In yet another embodiment, the invention comprises an antibody, or an antigen binding fragment thereof, wherein the antibody comprises a heavy chain variable region comprising the amino acid sequence of SEQ ID NO: 13 and a light chain variable region comprising the amino acid sequence of SEQ ID NO: 14; or a heavy chain variable region comprising the amino acid sequence of SEQ ID NO: 29 and a light chain variable region comprising the amino acid sequence of SEQ ID NO: 30; or a heavy chain variable region comprising the amino acid sequence of SEQ ID NO: 45 and a light chain variable region comprising the amino acid sequence of SEQ ID NO: 46, wherein the antibody neutralizes HIV-1 infection.

In a further embodiment, the invention comprises a recombinant antibody, antibody variant, or antigen binding fragment thereof, that neutralizes HIV-1 infection. In one embodiment, the antibody is not D5, a HR1-specific mAb antibody isolated from a human naïve B cell library (see Miller M. D. et al (2005) Proc Natl Acad Sci USA 102: 14759-14764).

In another aspect, the invention comprises a nucleic acid molecule comprising a polynucleotide encoding an antibody or antibody fragment of the invention that neutralizes HIV-1 infection. In yet another aspect, the invention comprises a cell expressing an antibody of the invention. In still another aspect, the invention comprises an isolated or purified immunogenic polypeptide comprising an epitope that binds to an antibody of the invention.

The invention also comprises a pharmaceutical composition comprising an antibody, an antibody variant or an antigen binding fragment of the invention, a nucleic acid of the invention, or an immunogenic polypeptide of the invention and a pharmaceutically acceptable diluent or carrier and, optionally, an agent useful for extending the half life of the antibody or antigen binding fragment thereof.

In another aspect of the invention, the invention provides a method of reducing HIV-1 infection or lowering the risk of HIV-1 infection or an HIV-1-related disease or a method of treating HIV-1 infection or an HIV-1-related disease. The method comprises administering to a subject in need thereof, a prophylactically or a therapeutically effective amount of at least one antibody, antibody variant, antigen binding fragment, or a pharmaceutical composition of the invention.

In yet another aspect of the invention, the invention comprises a method of screening for polypeptides that can induce or reveal an immune response against HIV-1, comprising screening polypeptide libraries using an antibody, an antibody fragment or variant of the invention.

In yet another aspect of the invention, the invention comprises a method of monitoring the quality of anti-HIV-1 vaccines. The method comprises using an antibody, an antibody variant, or an antigen binding fragment thereof of the invention to check that the antigen of the vaccine contains the specific epitope in the correct conformation.

In a further aspect of the invention, the invention comprises a vaccine comprising an epitope which specifically binds to an antibody, an antibody fragment or variant of the invention.

Use of an antibody of the invention, or an antigen binding fragment thereof, a nucleic acid of the invention, an immunogenic polypeptide of the invention, or a pharmaceutical composition of the invention (i) in the manufacture of a medicament for the treatment of HIV-1 infection, (ii) in a vaccine, or (iii) in diagnosis of HIV-1 infection is also contemplated to be within the scope of the invention. Further, use of an antibody of the invention, or an antigen binding fragment thereof, for monitoring the quality of anti-HIV-1 vaccines by checking that the antigen of said vaccine contains the specific epitope in the correct conformation is also contemplated to be within the scope of the invention.

In a further aspect, the invention comprises an epitope which specifically binds to an antibody of any one of the invention, or an antigen binding fragment thereof, for use (i) in therapy, (ii) in the manufacture of a medicament for treating HIV-1 infection, (iii) as a vaccine, or (iv) in screening for ligands able to neutralise HIV-1 infection.

### DESCRIPTION OF FIGURES

FIG. 1. Binding of gp120 and gp41-specific mAbs to a panel of 15 recombinant Env proteins from different clades. Different dilutions of mAbs were tested in ELISA against a panel of recombinant Env proteins representing 8 different clades. The mAbs are grouped according to the donor's virus clade. Shown is the half maximal binding concentration (K₅₀): red, 0.001-0.1 μg/ml, orange 0.1-5 μg/ml, yellow 5-500 μg/ml, white, no measurable binding.

FIG. 2. Neutralization of 20 HIV-1 isolates in HOS.CD4-R5 cells by a panel of human mAbs. (A) 46 mAbs (upper rows) were purified and tested in triplicates at a fixed concentration (100 μg/ml; *50 μg/ml and **25 μg/ml) for their capacity to neutralize 20 HIV-1 pseudoviruses (left columns) representing 6 different clades and both Tier-1 and Tier-2 isolates using HOS.CD4-R5 as target cells. Indicated is also the donor's HIV-1 clade. White, neutralization below 50%; yellow, 51-69%; orange, 70-89% and red, 90-100% neutralization. VSV-G pseudotyped HIV-1 was also tested as a negative control. (B) HK20, HGN194 and HJ16 were tested in parallel with b12, 2G12, 2F5 and 4E10 for their capacity to neutralize 20 HIV-1 pseudoviruses representing 6 different clades and both Tier-1 and Tier-2 isolates using HOS-CD4.R5 cells as in (A). Shown are IC50 values in μg/ml. -, indicates IC50 values >100 μg/ml.

FIG. 3. Neutralization of 92 HIV-1 isolates in TZM-bl cells by HK20, HGN194, HJ16. HK20, HGN194 and HJ16 were tested in parallel with b12, 2G12, 2F5, 4E10 and 447-52D for their capacity to neutralize 92 HIV-1 pseudoviruses representing 7 different clades and both Tier-1 and Tier-2 isolates using TZM-bl as target cells. Shown are IC50 values in μg/ml. -, indicates IC50 values >50 μg/ml, -* mAb tested starting from 25 μg/ml, nd, not determined. MuLV pseudotyped HIV-1 was also tested as a negative control.

FIG. 4. HJ16 binds to a CD4bs epitope distinct from that recognized by b12. (A) Binding of HJ16 and b12 to IIIB gp120 envelope protein. (B) Inhibition of IIIB gp120 binding to immobilized sCD4 by HJ16 and b12. (C-D) Inhibition of binding of HJ16 (C) or b12 (D) to immobilized gp120 by increasing concentrations of unlabeled HJ16 or b12. (E-F) Binding of b12 (E) or HJ16 (F) to YU2 wt gp120 and to the CD4i (I420R) or CD4bs (D368R) mutant YU2 proteins. Shown is mean±SD of triplicates.

FIG. 5. Epitope mapping of V3-specific mAbs by linear and circular peptide scanning. (A) Eight gp120-specific mAbs were mapped with linear and cyclic peptides to the V3 region. Shown is the HIV-1 isolate used for the mapping, the minimal epitope, the binding breadth expressed as number of recombinant Env proteins out of the 16 tested, and the fraction of isolates neutralized in the HOS and TZMbl-based neutralization assays, respectively. (B) Alignment of the region corresponding to the epitope recognized by mAb HGN194 in 44 HIV-1 isolates. Co-receptor binding residues are bold. Highlighted in grey are the isolates neutralized by HGN194 either in the TZMbl-based or in the HOS-based assays. (C) Replacement analysis at positions R(307), S(308), V(309), R(310), I(311), G(312), Q(315), T(316 and F(317) of the epitope recognized by HGN194. Shown is the binding to 92 peptides carrying various amino acid substitutions at positions critical for maintenance of MAb binding. The bars represent ELISA values of HGN194 binding with the wt peptide (dark patterned) and the variant peptide (black solid). The amino acid replacements corresponding to V3 sequences from all isolates shown in (B) are highlighted with light patterned bars. The binding of antibody to each peptide was tested in a PEPSCAN-based ELISA. Numbering according to HIV-1 HXB2.

FIG. 6. HK20 binds to the HR-1 region within gp41. (A) HK20 binding at 4 μg/ml to all overlapping linear peptides (15-mer peptides overlapping by 14 residues) spanning the gp41 sequence of the HXB2 isolate. Numbers at X-axis denote the first amino-terminal residue of the 15-mer gp41 peptide (numbering according to HIV-1 HXB2 sequence). Y— axis similar to FIG. 4C (B) Immunoprecipitation of HR-1, 5HB and HR-1-FP constructs in the presence (+) or absence (−) of HK20 mAb. Proteins were separated in 10% polyacrylamide gels under reducing conditions and stained with Coomassie blue. C, HK20 mAb alone; MW, molecular weight. (C) HK20 binding to gp41 constructs by ELISA. (D-E) Neutralization of 96ZM651.2 (D) and CH064.20 (E) HIV-1 primary isolates by HK20 IgG and Fab fragments in a HOS-based assay.

FIG. 7. Epitope specificity and neutralizing activity of the mAb panel. The pie chart shows the specificity of the 58 mAbs isolated from the 21 interrogated individuals. The fraction of antibodies with neutralizing activity against at least one isolate is indicated in parentheses. Partial CD4bs, incomplete inhibition of CD4 binding; HR-1/FP, epitope located in between HR-1 and the fusion peptide as determined by 5F3 competition; HR-1/5HB, recognition of trimeric HR-1 and 5-helix bundle; 5HB, 5 helix bundle; C-C region, gp41 immunodominant region; HR-2, heptad-repeat 2; ND, not defined epitopes within gp120 or gp41.

FIG. 8 shows the nucleotide and amino acid sequences of the heavy and light chains of HJ16.22. The CDR sequences are in bold.

FIG. 9 shows the nucleotide and amino acid sequences of the heavy and light chains of HK20.26. The CDR sequences are in bold.

FIG. 10 shows the nucleotide and amino acid sequences of the heavy and light chains of HGN194.12. The CDR sequences are in bold.

### DETAILED DESCRIPTION

The invention is based, in part, on the discovery of broadly neutralizing antibodies that neutralize HIV-1. Accordingly, in one aspect of the invention, the invention comprises a human antibody, an antibody variant, or an antigen binding fragment thereof, that neutralizes HIV-1.

As used herein, the terms “fragment,” “antibody fragment,” and “antigen binding fragment” are used interchangeably to refer to any fragment of an antibody of the invention that retains the antigen-binding activity of the antibody. Exemplary antibody fragments include, but are not limited to, Fab, Fab′, F(ab′)₂, Fv, and scFv fragments.

The terms “mutation,” and “substitution” are used interchangeably to refer to a change in one or more nucleic acid or amino acid residues.

As used herein, the terms “variant,” and “antibody variant” are used interchangeably to refer to any variant of an antibody of the invention that retains the antigen-binding activity of the antibodies. The term variant includes antibodies that comprise mutations and/or substitutions.

### Antibodies

The invention provides antibodies that neutralize HIV-1 infection. A “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used, alone or in combination, as prophylactic or therapeutic agents upon appropriate formulation, in association with active vaccination, as a diagnostic tool, or as a production tool as described herein.

In one embodiment, the invention comprises an antibody, a recombinant antibody, an antibody variant, or antigen binding fragments thereof, that neutralizes HIV-1 infection, wherein the antibody is not D5. D5 is a HR1-specific mAb antibody isolated from a human naïve B cell library (see Miller M. D. et al (2005) Proc Natl Acad Sci USA 102: 14759-14764).

In one embodiment, the antibodies of the invention preferentially neutralize infection of HIV-1 Tier 1 isolates. In another embodiment, the antibodies of the invention neutralize infection of HIV-1 Tier 2 and/or HIV-1 Tier 3 isolates. In yet another embodiment, the antibodies of the invention neutralize infection of HIV-1 Tier 1 and Tier 2 isolates. In yet another embodiment, the antibodies of the invention neutralize infection of HIV-1 Tier 1, Tier 2 and Tier 3 isolates. The antibodies of the invention may be isolated from clade B-infected individuals, or non-clade B-infected individuals, e.g., clade A- or clade C-infected individuals.

Without being bound to any specific theory, “Tier 1” as used herein refers to HIV isolates that are highly sensitive to antibody-mediated neutralization; “Tier 2” as used herein refers to HIV isolates that exhibit less sensitivity to neutralization as compared to Tier 1 isolates; and “Tier 3” as used herein refers to HIV isolates that exhibit more resistance (less sensitivity) to neutralization as compared to Tier 1 and Tier 2 isolates.

These antibodies, antigen binding fragment and variants can be used as prophylactic or therapeutic agents upon appropriate formulation, or as a diagnostic tool, as described herein. The antibodies of the invention may be monoclonal, for example, human monoclonal antibodies, or recombinant antibodies. The invention also provides fragments of the antibodies of the invention, particularly fragments that retain the antigen-binding activity of the antibodies. Although the specification, including the claims, may, in some places, refer explicitly to antibody fragment(s), variant(s) and/or derivative(s) of antibodies, it is understood that the term “antibody” or “antibody of the invention” includes all categories of antibodies, namely, antibody fragment(s), variant(s) and derivative(s) of antibodies.

The sequences of the heavy chains and light chains of several exemplary antibodies of the invention, each comprising three CDRs on the heavy chain and three CDRs on the light chain have been determined. The position of the CDR amino acids are defined according to the IMGT numbering system. The sequences of the CDRs, heavy chains, light chains as well as the sequences of the nucleic acid molecules encoding the CDRs, heavy chains, light chains of many exemplary antibodies of the invention are disclosed in the sequence listing. Table 1 provides the SEQ ID NOs for the amino acid sequences of the six CDRs, the variable region of the heavy and light chains, respectively, of exemplary antibodies of the invention. Table 2 provides the SEQ ID NOs for the sequences of the nucleic acid molecules encoding the CDRs, heavy chains and light chains of exemplary antibodies of the invention.

TABLE 1
Amino Acid SEQ IDs for Antibody CDRs, Heavy and Light Chains

| | | Heavy Chain | Light Chain |
|---|---|---|---|
| Antibody | CDRs | Variable Region | Variable Region |
| HJ16.22 | 1-6 | 13 | 14 |
| HK20.26 | 17-22 | 29 | 30 |
| HGN194.12 | 33-38 | 45 | 46 |

TABLE 2
Nucleic Acid SEQ IDs for Antibody CDRs, Heavy and Light Chains

| | | Heavy Chain | Light Chain |
|---|---|---|---|
| Antibody | CDRs | Variable Region | Variable Region |
| HJ16.22 | 7-12 | 15 | 16 |
| HK20.26 | 23-28 | 31 | 32 |
| HGN194.12 | 39-44 | 47 | 48 |

In one embodiment, the antibodies or antigen-binding fragments of the invention comprise one or more heavy or light chain CDRs of the exemplary antibodies of the invention. In an exemplary embodiment, the antibodies or antigen-binding fragments of the invention neutralize HIV-1 infection and comprise at least one CDR sequence having the sequence of any one of SEQ ID NOs: 1-6, 17-22, or 33-38.

In another embodiment, the antibodies, antibody variants or antigen binding fragments of the invention comprise a heavy chain comprising an amino acid sequence of one or more of SEQ ID NOs: 1-3, 17-19, or 33-35. In yet another embodiment, the antibodies, antibody variants or antigen binding fragments of the invention comprise a heavy chain CDR1 selected from the group consisting of SEQ ID NOs: 1, 17, and 33; a heavy chain CDR2 selected from the group consisting of SEQ ID NOs: 2, 18, and 34; and a heavy chain CDR3 selected from the group consisting of SEQ ID NOs: 3, 19, and 35.

For example, the antibodies of the invention, or fragments thereof, comprise a heavy chain comprising SEQ ID NO: 1 for CDRH1, SEQ ID NO: 2 for CDRH2, SEQ ID NO: 3 for CDRH3; SEQ ID NO: 17 for CDRH1, SEQ ID NO: 18 for CDRH2 and SEQ ID NO: 19 for CDRH3; SEQ ID NO: 33 for CDRH1, SEQ ID NO: 34 for CDRH2 and SEQ ID NO: 35 for CDRH3.

In yet another embodiment, the antibodies, antibody variants or antibody fragments of the invention comprise a light chain comprising an amino acid sequence of one or more of SEQ ID NOs: 4-6, 20-22, or 36-38. In a further embodiment, the antibodies, antibody variants or antibody fragments of the invention comprise a light chain CDR1 selected from the group consisting of SEQ ID NOs: 4, 20, and 36; a light chain CDR2 selected from the group consisting of SEQ ID NOs: 5, 21, and 37; and a light chain CDR3 selected from the group consisting of SEQ ID NOs: 6, 22, and 38.

For example, the antibodies of the invention comprise a light chain comprising SEQ ID NO: 4 for CDRL1, SEQ ID NO: 5 for CDRL2; SEQ ID NO: 6 for CDRL3; SEQ ID NO: 20 for CDRL1, SEQ ID NO: 21 for CDRL2; SEQ ID NO: 22 for CDRL3; SEQ ID NO: 36 for CDRL1, SEQ ID NO: 37 for CDRL2; SEQ ID NO: 38 for CDRL3.

In one embodiment, an antibody of the invention, or antigen binding fragment thereof, comprises all of the CDRs of antibody HJ16.22 as listed in Table 1, and neutralizes HIV-1 infection in a human host. In another embodiment, an antibody of the invention, or antigen binding fragment thereof, comprises all of the CDRs of antibody HK20.26 as listed in Table 1, and neutralizes HIV-1 infection in a human host. In another embodiment, an antibody of the invention, or antigen binding fragment thereof, comprises all of the CDRs of antibody HGN194.12 as listed in Table 1, and neutralizes HIV-1 infection in a human host.

In still another embodiment, the antibodies of the invention comprise a heavy chain with an amino acid sequence that is at least 70%, at least 75%, at least 80%, at least 85%, at least 90%, at least 95%, at least 97%, at least 98%, or at least 99% identical to those of SEQ ID NOs: 13, 29, or 45. In yet another embodiment, the antibodies of the invention comprise a light chain with an amino acid sequence that is at least 70%, at least 75%, at least 80%, at least 85%, at least 90%, at least 95%, at least 97%, at least 98%, or at least 99% identical to those of SEQ ID NOs: 14, 30, or 46.

In a further embodiment, the antibodies, antibody variants or antibody fragments of the invention comprise a heavy chain variable region comprising the amino acid sequence of any one of SEQ ID NOs: 13, 29, or 45, and a light chain variable region comprising the amino acid sequence of any one of SEQ ID NOs: 14, 30, or 46.

In yet another embodiment, the antibodies, antibody variants or antibody fragments of the invention neutralize HIV-1 infection and comprise a heavy chain variable region comprising the amino acid sequence of SEQ ID NO: 13 and a light chain variable region comprising the amino acid sequence of SEQ ID NO: 14; or a heavy chain variable region comprising the amino acid sequence of SEQ ID NO: 29 and a light chain variable region comprising the amino acid sequence of SEQ ID NO: 30; or a heavy chain variable region comprising the amino acid sequence of SEQ ID NO: 45 and a light chain variable region comprising the amino acid sequence of SEQ ID NO: 46.

Methods for chain replacement and for CDR grafting are well known in the art. Originally these methods were developed to humanize non-human antibodies (generally mouse antibodies) or to select human antibody counterparts having equivalent bioactivity to the non-human antibodies. These methods include replacement techniques where only one of the CDRs, for example, the CDR3s, of the non-human antibody are retained and the remainder of the V-region, including the framework and the other two CDRs, for example, the CDRs 1 and 2, are individually replaced in steps performed sequentially (e.g. U.S. Patent Application No. 20030166871; Rader, et al., Proc Natl Acad Sci USA 95:8910-15, 1998; Steinberg, et al., J Biol Chem 275:36073-78, 2000; Rader, et al., J Biol Chem 275:13668-76, 2000).

In addition, methods of creating antibodies with the binding specificities of a reference antibody for a target antigen are described in Patent Application No. WO05/069,970. The methods include transferring, from the reference antibody to a recipient antibody or antibody fragment, the minimal essential binding specificity of the reference antibody. Examples of regions that can be transferred include, but are not limited to, the transfer of a single CDR segment, for example a CDR3 segment, from the heavy and/or from the light chain, or a D segment, or a CDR3-FR4 segment, or any CDR3-FR4 segment that comprises the minimal essential binding specificity determinant. Antibodies created using these methods retain the binding specificity, and often affinity, of the reference antibody.

The antibodies, antibody variants or antibody fragments of the invention include antibodies that comprise, inter alia, one or more CDRs, a heavy chain or a light chain of an exemplary antibody of the invention and retain their specificity and ability to neutralize HIV-1 infection.

Examples of antibodies of the invention include, but are not limited to, HJ16.22, HK20.26 and HGN194.12 as described herein.

The invention further comprises an antibody, or fragment thereof, that binds to an epitope capable of binding to an antibody of the invention. The invention also comprises an antibody or an antibody fragment that competes with an antibody of the invention. In one embodiment, the antibody, or fragment thereof is not the D5 antibody described above.

The invention provides antibodies, antibody variants or antibody fragments thereof that specifically bind an epitope in a gp41 protein. In one embodiment, the antibodies and antibody fragments of the invention recognize and/or specifically bind an epitope in a gp41 protein that comprises His 564, Leu 565, Gln 567, Leu 568, Thr 569, Val 570, Trp 571, Ile 573, Lys 574, Gln 575, Gln 577, Ala 578, Ala 582 and His 643. The gp41 protein can be any gp41 protein. In one example it is the HXB2 gp41 reference protein sequence.

In another embodiment, heavy chain residues (using Kabat numbering) Ile 53, Phe 54, Asp 55, Ile 56, Asn 58, Ser 96, Tyr 97, Ser 98, Ser 99, Ser 100a, Pro 100b and Tyr 100c of the antibodies and antibody fragments of the invention are used in recognizing and/or binding the gp41 protein. In yet another embodiment, light chain residues (using Kabat numbering) Asp 93 and Leu 94 of the antibodies and antibody fragments of the invention are used in recognizing and/or binding the gp41 protein. In yet another embodiment, heavy chain residues Ile 53, Phe 54, Asp 55, Ile 56, Asn 58, Ser 96, Tyr 97, Ser 98, Ser 99, Ser 100a, Pro 100b and Tyr 100c and light chain residues Asp 93 and Leu 94 of the antibodies and antibody fragments of the invention are used in recognizing and/or binding the gp41 protein. In one embodiment, the antibody is not D5, as described above.

In another aspect, the invention also includes nucleic acid sequences encoding part or all of the light and heavy chains and CDRs of the antibodies of the present invention. In one embodiment, nucleic acid sequences according to the invention include nucleic acid sequences having at least 70%, at least 75%, at least 80%, at least 85%, at least 90%, at least 95%, at least 98%, or at least 99% identity to the nucleic acid encoding a heavy or light chain of an antibody of the invention. In another embodiment, a nucleic acid sequence of the invention has the sequence of a nucleic acid encoding a heavy or light chain CDR of an antibody of the invention. For example, a nucleic acid sequence according to the invention comprises a sequence that is at least 70%, at least 75%, at least 80%, at least 85%, at least 90%, at least 95%, at least 98%, or at least 99% identical to the nucleic acid sequences of SEQ ID NOs: 7-12, 15-16, 23-28, 31-32, 39-44, or 47-48.

Due to the redundancy of the genetic code, variants of these sequences will exist that encode the same amino acid sequences. These variants are included within the scope of the invention.

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code, as mentioned above or may be produced due to errors in transcription or translation. Variants may also be introduced to modify the antibody effector function, for instance in the Fc region to enhance or reduce the binding of the antibody to an Fc receptor.

Further variants of the antibody sequences having improved affinity and/or potency may be obtained using methods known in the art and are included within the scope of the invention. For example, amino acid substitutions may be used to obtain antibodies with further improved affinity. Alternatively, codon optimisation of the nucleotide sequence may be used to improve the efficiency of translation in expression systems for the production of the antibody. Further, polynucleotides comprising a sequence optimized for antibody specificity or neutralizing activity by the application of a directed evolution method to any of the nucleic acid sequences of the invention are also within the scope of the invention.

In one embodiment variant antibody sequences may share 70% or more (i.e. 75%, 80%, 85%, 90%, 95%, 97%, 98%, 99% or more) amino acid sequence identity with the sequences recited in the application. In some embodiments such sequence identity is calculated with regard to the full length of the reference sequence (i.e., the sequence recited in the application). In some further embodiments, percentage identity, as referred to herein, is as determined using BLAST version 2.1.3 using the default parameters specified by the NCBI (the National Center for Biotechnology Information) [Blosum 62 matrix; gap open penalty=11 and gap extension penalty=1].

Further included within the scope of the invention are vectors, for example expression vectors, comprising a nucleic acid sequence according to the invention. Cells transformed with such vectors are also included within the scope of the invention. Examples of such cells include but are not limited to, eukaryotic cells, e.g., yeast cells, animal cells or plant cells. In one embodiment the cells are mammalian, e.g., human, EBV-immortalized B cells, CHO, HEK293T, PER.C6, NS0, myeloma or hybridoma cells.

The invention also relates to monoclonal antibodies that bind to an epitope capable of binding an antibody of the invention. In one embodiment, the invention includes a monoclonal antibody that binds to an epitope capable of binding a monoclonal antibody selected from the group consisting of HJ16.22, HK20.26 and HGN194.12.

Monoclonal and recombinant antibodies are particularly useful in identification and purification of the individual polypeptides or other antigens against which they are directed. The antibodies of the invention have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme. The antibodies may also be used for the molecular identification and characterisation (epitope mapping) of antigens.

Antibodies of the invention will typically be glycosylated. N-linked glycans attached to the CH2 domain of a heavy chain, for instance, can influence C1q and FcR binding, with aglycosylated antibodies having lower affinity for these receptors. The glycan structure can also affect activity e.g., differences in complement-mediated cell death may be seen depending on the number of galactose sugars (0, 1 or 2) at the terminus of a glycan's biantennary chain. An antibody's glycans preferably do not lead to a human immunogenic response after administration.

Antibodies of the invention can be coupled to a drug for delivery to a treatment site or coupled to a detectable label to facilitate imaging of a site comprising cells of interest, such as cells infected with HIV-1. Methods for coupling antibodies to drugs and detectable labels are well known in the art, as are methods for imaging using detectable labels. Labelled antibodies may be employed in a wide variety of assays, employing a wide variety of labels. Detection of the formation of an antibody-antigen complex between an antibody of the invention and an epitope of interest can be facilitated by attaching a detectable substance to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or ³H. Such labeled reagents may be used in a variety of well-known assays, such as radioimmunoassays, enzyme immunoassays, e.g., ELISA, fluorescent immunoassays, and the like.

An antibody according to the invention may be conjugated to a therapeutic moiety such as a cytotoxin, a therapeutic agent, or a radioactive metal ion or radioisotope. Examples of radioisotopes include, but are not limited to, I-131, I-123, I-125, Y-90, Re-188, Re-186, At-211, Cu-67, Bi-212, Bi-213, Pd-109, Tc-99, In-111, and the like. Such antibody conjugates can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, calicheamicin bacterial toxin, or diphtheria toxin.

Techniques for conjugating such therapeutic moiety to antibodies are well known. See, for example, Arnon et al. (1985) “Monoclonal Antibodies for Immunotargeting of Drugs in Cancer Therapy,” in Monoclonal Antibodies and Cancer Therapy, ed. Reisfeld et al. (Alan R. Liss, Inc.), pp. 243-256; ed. Hellstrom et al. (1987) “Antibodies for Drug Delivery,” in Controlled Drug Delivery, ed. Robinson et al. (2d ed; Marcel Dekker, Inc.), pp. 623-653; Thorpe (1985) “Antibody Carriers of Cytotoxic Agents in Cancer Therapy: A Review,” in Monoclonal Antibodies '84: Biological and Clinical Applications, ed. Pinchera et al. pp. 475-506 (Editrice Kurtis, Milano, Italy, 1985); “Analysis, Results, and Future Prospective of the Therapeutic Use of Radiolabeled Antibody in Cancer Therapy,” in Monoclonal Antibodies for Cancer Detection and Therapy, ed. Baldwin et al. (Academic Press, New York, 1985), pp. 303-316; and Thorpe et al. (1982) Immunol. Rev. 62:119-158.

Alternatively, an antibody can be conjugated to a second antibody to form an antibody heteroconjugate as described in U.S. Pat. No. 4,676,980. In addition, linkers may be used between the labels and the antibodies of the invention (see U.S. Pat. No. 4,831,175). Antibodies or, antigen-binding fragments thereof may be directly labeled with radioactive iodine, indium, yttrium, or other radioactive particle known in the art (see U.S. Pat. No. 5,595,721). Treatment may consist of a combination of treatment with conjugated and non-conjugated antibodies administered simultaneously or subsequently (see WO00/52031 and WO00/52473). Antibodies of the invention may also be attached to a solid support.

Additionally, antibodies of the invention, or functional antibody fragments thereof, can be chemically modified by covalent conjugation to a polymer to, for example, increase their circulating half-life, for example. Examples of polymers, and methods to attach them to peptides, are shown in U.S. Pat. No. 4,766,106, U.S. Pat. No. 4,179,337, U.S. Pat. No. 4,495,285 and U.S. Pat. No. 4,609,546. In some embodiments the polymers may be selected from polyoxyethylated polyols and polyethylene glycol (PEG). PEG is soluble in water at room temperature and has the general formula: R(O—CH₂—CH₂)ₙO—R where R can be hydrogen, or a protective group such as an alkyl or alkanol group. In one embodiment the protective group may have between 1 and 8 carbons. In a further embodiment the protective group is methyl. The symbol n is a positive integer. In one embodiment n is between 1 and 1,000. In another embodiment n is between 2 and 500. In one embodiment the PEG has an average molecular weight between 1,000 and 40,000. In a further embodiment the PEG has a molecular weight between 2,000 and 20,000. In yet a further embodiment the PEG has a molecular weight of between 3,000 and 12,000. In one embodiment PEG has at least one hydroxy group. In another embodiment the PEG has a terminal hydroxy group. In yet another embodiment it is the terminal hydroxy group which is activated to react with a free amino group on the inhibitor. However, it will be understood that the type and amount of the reactive groups may be varied to achieve a covalently conjugated PEG/antibody of the present invention.

Antibodies of the invention can be modified by introducing random amino acid mutations into particular region of the CH2 or CH3 domain of the heavy chain in order to alter their binding affinity for FcRn and/or their serum half-life in comparison to the unmodified antibodies. Examples of such modifications include, but are not limited to, substitutions of at least one amino acid from the heavy chain constant region selected from the group consisting of amino acid residues 250, 314, and 428.

Water-soluble polyoxyethylated polyols are also useful in the present invention. They include polyoxyethylated sorbitol, polyoxyethylated glucose, polyoxyethylated glycerol (POG), and the like. In one embodiment, POG is used. Without being bound by any theory, because the glycerol backbone of polyoxyethylated glycerol is the same backbone occurring naturally in, for example, animals and humans in mono-, di-, triglycerides, this branching would not necessarily be seen as a foreign agent in the body. In some embodiments POG has a molecular weight in the same range as PEG. The structure for POG is shown in Knauf et al. (1988) J. Bio. Chem. 263:15064-15070, and a discussion of POG/IL-2 conjugates is found in U.S. Pat. No. 4,766,106.

Another drug delivery system that can be used for increasing circulatory half-life is the liposome. Methods of preparing liposome delivery systems are discussed in Gabizon et al. (1982) Cancer Research 42:4734; Cafiso (1981) Biochem Biophys Acta 649:129; and Szoka (1980) Ann. Rev. Biophys. Eng. 9:467. Other drug delivery systems are known in the art and are described in, for example, Poznansky et al. (1980) Drug Delivery Systems (R. L. Juliano, ed., Oxford, N.Y.) pp. 253-315 and Poznansky (1984) Pharm Revs 36:277.

Antibodies of the invention may be provided in purified form. Typically, the antibody will be present in a composition that is substantially free of other polypeptides e.g. where less than 90% (by weight), usually less than 60% and more usually less than 50% of the composition is made up of other polypeptides.

Antibodies of the invention may be immunogenic in non-human (or heterologous) hosts e.g. in mice. In particular, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanisation or from xeno-mice.

Antibodies of the invention can be of any isotype (e.g., IgA, IgG, IgM i.e. an α, γ or μ heavy chain), but will generally be IgG. Within the IgG isotype, antibodies may be IgG1, IgG2, IgG3 or IgG4 subclass. In one embodiment, the antibody is IgG1. Antibodies of the invention may have a κ or a λ light chain.

Included within the scope of the invention are HIV-1-neutralizing recombinant or engineered bispecific antibody molecules or antigen binding fragments thereof. Such antibodies and fragments may comprise a first binding site for an epitope on a first HIV-1 and a second binding site for a second epitope on the same HIV-1 or on a different virus. The variable domains of the respective binding sites can be formed as immunoglobulin isotypes of the invention or as heterodimeric Fab, Fab′, F(ab′)2, ScFv or diabodies that can be linked together via one or more peptide linkers.

### Production of Antibodies

Monoclonal antibodies according to the invention can be made by any method known in the art. The general methodology for making monoclonal antibodies using hybridoma technology is well known (see Kohler, G. and Milstein, C,. 1975, Nature 256:495-497; and Kozbar et al. 1983, Immunology Today 4:72). Preferably, the alternative EBV immortalisation method described in WO2004/076677 is used.

Using the method described in WO2004/076677, B cells producing the antibody of the invention can be transformed with EBV in the presence of a polyclonal B cell activator. Transformation with EBV is a standard technique and can easily be adapted to include polyclonal B cell activators.

Additional stimulants of cellular growth and differentiation may optionally be added during the transformation step to further enhance the efficiency. These stimulants may be cytokines such as IL-2 and IL-15. In one aspect, IL-2 is added during the immortalisation step to further improve the efficiency of immortalisation, but its use is not essential.

The immortalised B cells produced using these methods can then be cultured using methods known in the art and antibodies isolated therefrom.

The antibodies of the invention can also be made by culturing plasma cells in microwell culture plates using the method described in UK Patent Application 0819376.5, U.S. application Ser. No. 12/604,240 and PCT publication No. WO2010/046775. Further, from plasma cell cultures, RNA can be extracted and PCR can be performed using methods known in the art. The VH and VL regions of the antibodies can be amplified by RT-PCR, sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

Monoclonal antibodies may be further purified, if desired, using filtration, centrifugation and various chromatographic methods such as HPLC or affinity chromatography. Techniques for purification of monoclonal antibodies, including techniques for producing pharmaceutical-grade antibodies, are well known in the art.

Fragments of the monoclonal antibodies of the invention can be obtained from the monoclonal antibodies by methods that include digestion with enzymes, such as pepsin or papain, and/or by cleavage of disulfide bonds by chemical reduction. Alternatively, fragments of the monoclonal antibodies can be obtained by cloning and expression of part of the sequences of the heavy or light chains. Antibody “fragments” may include Fab, Fab′, F(ab′)₂ and Fv fragments. The invention also encompasses single-chain Fv fragments (scFv) derived from the heavy and light chains of a monoclonal antibody of the invention e.g. the invention includes a scFv comprising the CDRs from an antibody of the invention. Also included are heavy or light chain monomers and dimers as well as single chain antibodies, e.g. single chain Fv in which the heavy and light chain variable domains are joined by a peptide linker.

Standard techniques of molecular biology may be used to prepare DNA sequences coding for the antibodies or fragments or variants of the antibodies of the present invention. Desired DNA sequences may be synthesised completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention or fragments thereof. Bacterial, for example E. coli, and other microbial systems may be used, in part, for expression of antibody fragments such as Fab and F(ab′)₂ fragments, and especially Fv fragments and single chain antibody fragments, for example, single chain Fvs. Eukaryotic, e.g. mammalian, host cell expression systems may be used for production of larger antibody molecules, including complete antibody molecules. Suitable mammalian host cells include CHO, HEK293T, PER.C6, NS0, myeloma or hybridoma cells.

The present invention also provides a process for the production of an antibody of the invention comprising culturing a host cell comprising a vector of the present invention under conditions suitable for leading to expression of protein from DNA encoding the antibody of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Alternatively, antibodies according to the invention may be produced by i) expressing a nucleic acid sequence according to the invention in a cell, and ii) isolating the expressed antibody product. Additionally, the method may include iii) purifying the antibody.

### Screening and Isolation of B Cells

Transformed B cells may be screened for those producing antibodies of the desired antigen specificity, and individual B cell clones may then be produced from the positive cells.

The screening step may be carried out by ELISA, by staining of tissues or cells (including infected or transfected cells), a neutralisation assay or one of a number of other methods known in the art for identifying desired antigen specificity. The assay may select on the basis of simple antigen recognition, or may select on the additional basis of a desired function e.g. to select neutralizing antibodies rather than just antigen-binding antibodies, to select antibodies that can change characteristics of targeted cells, such as their signalling cascades, their shape, their growth rate, their capability of influencing other cells, their response to the influence by other cells or by other reagents or by a change in conditions, their differentiation status, etc.

The cloning step for separating individual clones from the mixture of positive cells may be carried out using limiting dilution, micromanipulation, single cell deposition by cell sorting or another method known in the art.

The immortalised B cell clones of the invention can be used in various ways e.g. as a source of monoclonal antibodies, as a source of nucleic acid (DNA or mRNA) encoding a monoclonal antibody of interest, for research, etc.

The invention provides a composition comprising immortalised B memory cells, wherein the cells produce antibodies that neutralize HIV-1 infection, and wherein the antibodies are produced at >5 pg per cell per day. The invention also provides a composition comprising clones of an immortalised B memory cell, wherein the clones produce a monoclonal antibody that neutralizes HIV-1 infection, and wherein the antibody is produced at >5 pg per cell per day.

Exemplary immortalised B cell clone according to the invention include, but are not limited to, HJ16.22, HK20.26 and HGN194.12.

### Epitopes

As mentioned above, the antibodies of the invention can be used to map the epitopes to which they bind. The epitopes recognised by the antibodies of the present invention may have a number of uses. The epitope and mimotopes thereof in purified or synthetic form can be used to raise immune responses (i.e. as a vaccine, or for the production of antibodies for other uses) or for screening patient serum for antibodies that immunoreact with the epitope or mimotopes thereof. In one embodiment such an epitope or mimotope, or antigen comprising such an epitope or mimotope may be used as a vaccine for raising an immune response. The antibodies and antigen binding fragments of the invention can also be used in a method of monitoring the quality of vaccines. In particular the antibodies can be used to check that the antigen in a vaccine contains the specific epitope in the correct conformation.

The epitope may also be useful in screening for ligands that bind to said epitope. Such ligands, include but are not limited to antibodies, including those from camels, sharks and other species, fragments of antibodies, peptides, phage display technology products, aptamers, adnectins, synthetic compounds, or fragments of other viral or cellular proteins, that may block the epitope and so prevent infection. Such ligands are encompassed within the scope of the invention.

In one embodiment, the antibodies, antibody variants or antibody fragments of the invention specifically bind an epitope on the HIV-1 gp41 protein as described above. The epitope comprises residues His 564, Leu 565, Gln 567, Leu 568, Thr 569, Val 570, Trp 571, Ile 573, Lys 574, Gln 575, Gln 577, Ala 578, Ala 582 and His 643 of the HXB2 gp41 reference protein sequence.

### Recombinant Expression

The immortalised B memory cells of the invention may also be used as a source of nucleic acid for the cloning of antibody genes for subsequent recombinant expression. Expression from recombinant sources is more common for pharmaceutical purposes than expression from B cells or hybridomas e.g. for reasons of stability, reproducibility, culture ease, etc.

Thus the invention provides a method for preparing a recombinant cell, comprising the steps of: (i) obtaining one or more nucleic acids (e.g. heavy and/or light chain genes) from the B cell clone that encodes the antibody of interest; and (ii) inserting the nucleic acid into an expression host in order to permit expression of the antibody of interest in that host.

Similarly, the invention provides a method for preparing a recombinant cell, comprising the steps of: (i) sequencing nucleic acid(s) from the B cell clone that encodes the antibody of interest; and (ii) using the sequence information from step (i) to prepare nucleic acid(s) for insertion into an expression host in order to permit expression of the antibody of interest in that host. The nucleic acid may, but need not, be manipulated between steps (i) and (ii) to introduce restriction sites, to change codon usage, to optimise transcription and/or translation regulatory sequences, and/or to modify effector function.

The invention also provides a method of preparing a recombinant cell, comprising the step of transforming a host cell with one or more nucleic acids that encode a monoclonal antibody of interest, wherein the nucleic acids are nucleic acids that were derived from an immortalised B cell clone of the invention. Thus the procedures for first preparing the nucleic acid(s) and then using it to transform a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture techniques can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells are well known in the art (e.g., Chapter 4 of Kuby Immunology (4th edition, 2000; ASIN: 0716733315)).

The expression host is preferably a eukaryotic cell, including yeast and animal cells, particularly mammalian cells. Examples of cells include, but are not limited to, CHO cells, NS0 cells, human cells such as PER.C6 (Crucell; Jones et al. Biotechnol Prog 2003, 19(1):163-8); HKB-11 cells (Bayer; Cho et al. Cytotechnology 2001, 37:23-30; and Cho et al. Biotechnol Prog 2003, 19:229-32); myeloma cells (U.S. Pat. No. 5,807,715 and U.S. Pat. No. 6,300,104), etc.), as well as plant cells. Preferred expression hosts can glycosylate the antibody of the invention, particularly with carbohydrate structures that are not themselves immunogenic in humans. In one embodiment the expression host may be able to grow in serum-free media. In a further embodiment the expression host may be able to grow in culture without the presence of animal-derived products. The expression host may be cultured to give a cell line.

The invention provides a method for preparing one or more nucleic acid molecules (e.g. heavy and light chain genes) that encode an antibody of interest, comprising the steps of: (i) preparing an immortalised B cell clone according to the invention; (ii) obtaining from the B cell clone nucleic acid that encodes the antibody of interest. The invention also provides a method for obtaining a nucleic acid sequence that encodes an antibody of interest, comprising the steps of: (i) preparing an immortalised B cell clone according to the invention; (ii) sequencing nucleic acid from the B cell clone that encodes the antibody of interest.

The invention also provides a method of preparing nucleic acid molecule(s) that encodes an antibody of interest, comprising the step of obtaining the nucleic acid from a B cell clone that was obtained from a transformed B cell of the invention. Thus the procedures for first obtaining the B cell clone and then preparing nucleic acid(s) from it can be performed at very different times by different people in different places (e.g. in different countries).

The invention provides a method for preparing an antibody (e.g. for pharmaceutical use), comprising the steps of: (i) obtaining and/or sequencing one or more nucleic acids (e.g. heavy and light chain genes); (ii) using the sequence information from step (i) to prepare nucleic acid(s) for insertion into an expression host in order to permit expression of the antibody of interest in that host; (iii) culturing or sub-culturing the expression host under conditions where the antibody of interest is expressed; and, optionally, (iv) purifying the antibody of the interest. The nucleic acid can, but need not be, obtained and/or sequenced from a B cell clone expressing the antibody of interest. In one embodiment, the nucleic acid from step (i) may, optionally be modified so as to introduce desired substitutions in the amino acid sequence of the antibody.

The invention also provides a method of preparing an antibody comprising the steps of: culturing or sub-culturing an expression host cell population under conditions where the antibody of interest is expressed and, optionally, purifying the antibody of the interest, wherein said expression host cell population has been prepared by (i) providing nucleic acid(s) encoding an antibody of interest; (ii) inserting the nucleic acid(s) into an expression host that can express the antibody of interest, and (iii) culturing or sub-culturing expression hosts comprising said inserted nucleic acids to produce said expression host cell population.

### Pharmaceutical Compositions

The invention provides a pharmaceutical composition comprising an antibody and/or antibody fragment of the invention and/or nucleic acid encoding such antibodies and/or immortalised B cells that express such antibodies and/or the epitopes recognised by the antibodies of the invention. A pharmaceutical composition may also contain a pharmaceutically acceptable carrier to allow administration. The carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Within the scope of the invention, forms of administration may include those forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the invention can be administered directly to the subject. In one embodiment the compositions are adapted for administration to human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. Dosage treatment may be a single dose schedule or a multiple dose schedule. Known antibody-based pharmaceuticals provide guidance relating to frequency of administration e.g. whether a pharmaceutical should be delivered daily, weekly, monthly, etc. Frequency and dosage may also depend on the severity of symptoms.

Compositions of the invention may be prepared in various forms. For example, the compositions may be prepared as injectables, either as liquid solutions or suspensions. Solid forms suitable for solution, or suspension, in liquid vehicles prior to injection can also be prepared (e.g. a lyophilised composition, like Synagis™ and Herceptin™, for reconstitution with sterile water containing a preservative). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. For example, a lyophilised antibody can be provided in kit form with sterile water or a sterile buffer.

It will be appreciated that the active ingredient in the composition will be an antibody molecule, an antibody fragment or variants and derivatives thereof. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments this may be between 6 and 8, and in further embodiments about 7. The pH may be maintained by the use of a buffer. The composition may be sterile and/or pyrogen free. The composition may be isotonic with respect to humans. In one embodiment pharmaceutical compositions of the invention are supplied in hermetically-sealed containers.

Pharmaceutical compositions will include a therapeutically effective amount of one or more antibodies of the invention and/or a polypeptide comprising an epitope that binds an antibody of the invention i.e. an amount that is sufficient to treat, ameliorate, or prevent a desired disease or condition, or to exhibit a detectable therapeutic effect. Therapeutic effects also include reduction in physical symptoms. The precise effective amount for any particular subject will depend upon their size and health, the nature and extent of the condition, and the therapeutics or combination of therapeutics selected for administration. The effective amount for a given situation is determined by routine experimentation and is within the judgment of a clinician. For purposes of the present invention, an effective dose will generally be from about 0.01 mg/kg to about 50 mg/kg, or about 0.05 mg/kg to about 10 mg/kg of the compositions of the present invention in the individual to which it is administered. Known antibody-based pharmaceuticals provide guidance in this respect e.g., Herceptin™ is administered by intravenous infusion of a 21 mg/ml solution, with an initial loading dose of 4 mg/kg body weight and a weekly maintenance dose of 2 mg/kg body weight; Rituxan™ is administered weekly at 375 mg/m²; etc.

In one embodiment pharmaceutical compositions can include more than one antibody of the invention. In another embodiment the composition comprises two or more antibodies, wherein the first antibody is specific for a first HIV-1 epitope, and the second antibody is specific for a second, different HIV-1 epitope. In yet another embodiment, the pharmaceutical composition comprises three antibodies of the invention.

In one embodiment, the invention provides a pharmaceutical composition comprising the antibody HJ16.22 or an antigen binding fragment thereof, and a pharmaceutically acceptable diluent or carrier. In another embodiment, the invention provides a pharmaceutical composition comprising the antibody HK20.26 or an antigen binding fragment thereof, and a pharmaceutically acceptable diluent or carrier. In another embodiment, the invention provides a pharmaceutical composition comprising the antibody HGN194.12 or an antigen binding fragment thereof, and a pharmaceutically acceptable diluent or carrier.

Antibodies of the invention may be administered (either combined or separately) with other therapeutics e.g. with chemotherapeutic compounds, with radiotherapy, etc. Preferred therapeutic compounds include anti-viral compounds. Such combination therapy provides an additive or synergistic improvement in therapeutic efficacy relative to the individual therapeutic agents when administered alone. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In compositions of the invention that include antibodies of the invention, the antibodies may make up at least 50% by weight (e.g. 60%, 70%, 75%, 80%, 85%, 90%, 95%, 97%, 98%, 99% or more) of the total protein in the composition. The antibodies are thus in purified form.

The invention provides a method of preparing a pharmaceutical, comprising the steps of: (i) preparing an antibody of the invention; and (ii) admixing the purified antibody with one or more pharmaceutically-acceptable carriers.

The invention also provides a method of preparing a pharmaceutical, comprising the step of admixing an antibody with one or more pharmaceutically-acceptable carriers, wherein the antibody is a monoclonal antibody that was obtained from a transformed B cell of the invention. Thus the procedures for first obtaining the monoclonal antibody and then preparing the pharmaceutical can be performed at very different times by different people in different places (e.g. in different countries).

As an alternative to delivering antibodies for therapeutic purposes, it is possible to deliver nucleic acid (typically DNA) that encodes the monoclonal antibody (or active fragment thereof) of interest to a subject, such that the nucleic acid can be expressed in the subject in situ to provide a desired therapeutic effect. Suitable gene therapy and nucleic acid delivery vectors are known in the art.

Compositions of the invention may be immunogenic compositions, and in some embodiments may be vaccine compositions comprising an antigen comprising a HIV-1 epitope. Vaccines according to the invention may either be prophylactic (i.e. to prevent infection) or therapeutic (i.e. to treat infection).

Compositions may include an antimicrobial, particularly if packaged in a multiple dose format. Compositions may comprise detergent e.g. a Tween (polysorbate), such as Tween 80. Detergents are generally present at low levels e.g. <0.01%. Compositions may include sodium salts (e.g. sodium chloride) to give tonicity. A concentration of 10±2 mg/ml NaCl is typical.

Compositions may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material. The pH of a composition for lyophilisation may be adjusted to around 6.1 prior to lyophilisation.

The compositions of the invention may also comprise one or more immunoregulatory agents. In one embodiment, one or more of the immunoregulatory agents include(s) an adjuvant.

### Medical Treatments and Uses

The antibodies, antigen binding fragments, derivatives and variants thereof, or the pharmaceutical compositions of the invention can be used for the treatment of HIV-1 infection, for the prevention of HIV-1 infection or for the diagnosis of HIV-1 infection.

Methods of diagnosis, as further described below, may include contacting an antibody or an antibody fragment with a sample. Such samples may be tissue samples taken from, for example, salivary glands, lung, liver, pancreas, kidney, ear, eye, placenta, alimentary tract, heart, ovaries, pituitary, adrenals, thyroid, brain or skin. In one embodiment, the sample may be a blood sample.

The invention therefore provides (i) an antibody, an antibody fragment, or variants and derivatives thereof according to the invention, (ii) an immortalised B cell clone according to the invention, (iii) an epitope capable of binding an antibody of the invention or (iv) a ligand, preferably an antibody, capable of binding an epitope that binds an antibody of the invention for use in therapy.

Also provided is a method of treating a subject comprising administering to that subject (i) an antibody, an antibody fragment, variants and derivatives thereof, or a pharmaceutical composition according to the invention, or, a ligand, preferably an antibody, capable of binding an epitope that binds an antibody of the invention.

The invention also provides the use of (i) an antibody, an antibody fragment, or variants and derivatives thereof according to the invention, (ii) an immortalised B cell clone according to the invention, (iii) an epitope capable of binding an antibody of the invention, or (iv) a ligand, preferably an antibody, that binds to an epitope capable of binding an antibody of the invention, in the manufacture of a medicament for the prevention or treatment of HIV-1 infection.

The invention provides a pharmaceutical composition for use as a medicament for the prevention or treatment of HIV-1 infection. It also provides the use of an antibody of the invention and/or a protein comprising an epitope to which such an antibody binds in the manufacture of a medicament for treatment of a patient and/or diagnosis in a patient. It also provides a method for treating a subject, e.g., a human subject. The method comprises the step of administering to the subject a therapeutically effective dose of a composition of the invention. One way of checking efficacy of therapeutic treatment involves monitoring disease symptoms after administration of the composition of the invention. Treatment can be a single dose schedule or a multiple dose schedule.

In one embodiment, an antibody, antibody fragment, antibody variant, epitope or pharmaceutical composition according to the invention is administered to a subject in need of such treatment. Such a subject includes, but is not limited to, one who is particularly at risk of or susceptible to HIV-1 infection.

Antibodies of the invention can be used in passive immunisation as further described below. Antibodies and fragments or variants thereof, or a nucleic acid encoding an antibody or an antibody fragment or variant as described in the present invention may also be used in a kit for the diagnosis of HIV-1 infection.

Epitopes capable of binding an antibody of the invention, e.g., the monoclonal antibodies HJ16.22, HK20.26, or HGN194.12, may be used in a kit for monitoring the efficacy of vaccination procedures by detecting the presence of protective anti-HIV-1 antibodies. In one embodiment, the invention provides a kit comprising gp41 protein or polypeptide that comprises the epitope recognized by the antibodies or antibody fragments of the invention.

Antibodies, antibody fragments, or variants and derivatives thereof, as described in the present invention may also be used in a kit for monitoring vaccine manufacture with the desired immunogenicity.

The invention also provides a method of preparing a pharmaceutical composition, comprising the step of admixing a monoclonal antibody with one or more pharmaceutically-acceptable carriers, wherein the monoclonal antibody is a monoclonal antibody that was obtained from an expression host of the invention. Thus the procedures for first obtaining the monoclonal antibody (e.g. expressing it and/or purifying it) and then admixing it with the pharmaceutical carrier(s) can be performed at very different times by different people in different places (e.g. in different countries).

Starting with a transformed B cell of the invention, various steps of culturing, sub-culturing, cloning, sub-cloning, sequencing, nucleic acid preparation etc. can be performed in order to perpetuate the antibody expressed by the transformed B cell, with optional optimisation at each step. In one embodiment, the above methods further comprise techniques of optimisation (e.g. affinity maturation or optimisation) applied to the nucleic acids encoding the antibody. The invention encompasses all cells, nucleic acids, vectors, sequences, antibodies etc. used and prepared during such steps.

In all these methods, the nucleic acid used in the expression host may be manipulated to insert, delete or amend certain nucleic acid sequences. Changes from such manipulation include, but are not limited to, changes to introduce restriction sites, to amend codon usage, to add or optimise transcription and/or translation regulatory sequences, etc. It is also possible to change the nucleic acid to alter the encoded amino acids. For example, it may be useful to introduce one or more (e.g. 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g. labels) or can introduce tags (e.g. for purification purposes). Mutations can be introduced in specific sites or can be introduced at random, followed by selection (e.g. molecular evolution). For instance, one or more nucleic acids encoding any of the CDR regions, heavy chain variable regions or light chain variable regions of antibodies of the invention can be randomly or directionally mutated to introduce different properties in the encoded amino acids. Such changes can be the result of an iterative process wherein initial changes are retained and new changes at other nucleotide positions are introduced. Moreover, changes achieved in independent steps may be combined. Different properties introduced into the encoded amino acids may include, but are not limited to, enhanced affinity.

### Diagnostic Assays

Antibodies provided herein can be detected by appropriate assays, e.g., conventional types of immunoassays. For example, a sandwich assay can be performed in which a HIV-1 protein (e.g., Env) or fragment thereof is affixed to a solid phase. Incubation is maintained for a sufficient period of time to allow the antibody in the sample to bind to the immobilized polypeptide on the solid phase. After this first incubation, the solid phase is separated from the sample. The solid phase is washed to remove unbound materials and interfering substances such as non-specific proteins which may also be present in the sample. The solid phase containing the antibody of interest (e.g. monoclonal antibody HK20.26, HJ16.22 or HGN194.12) bound to the immobilized polypeptide is subsequently incubated with a second, labeled antibody or antibody bound to a coupling agent such as biotin or avidin. This second antibody may be another anti-HIV-1 antibody or another antibody. Labels for antibodies are well-known in the art and include radionuclides, enzymes (e.g. maleate dehydrogenase, horseradish peroxidase, glucose oxidase, and catalase), fluors (fluorescein isothiocyanate, rhodamine, phycocyanin, and fluorescarmine), biotin, and the like. The labeled antibodies are incubated with the solid and the label bound to the solid phase is measured. These and other immunoassays can be easily performed by those of ordinary skill in the art.

In one example, a method for detecting the presence or absence of HIV-1 in a biological sample involves obtaining a biological sample from a test subject and contacting the biological sample with a labeled monoclonal or scFv antibody according to the invention such that the presence of the virus is detected in the biological sample.

As used herein, the term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method provided herein can be used to detect HIV-1 in a biological sample in vitro as well as in vivo. For example, in vitro techniques for detection of HIV-1 include enzyme linked immunosorbent assays (ELISAs), Western blots, immunoprecipitations, and immunofluorescence. Furthermore, in vivo techniques for detection of HIV-1 include introducing into a subject a labeled anti-HIV-1 antibody. For example, the antibody can be labeled with a radioactive marker whose presence and location in a subject can be detected by standard imaging techniques.

In one embodiment, the biological sample contains protein molecules from the subject. One preferred biological sample is a peripheral blood leukocyte sample isolated by conventional means from a subject.

The invention also encompasses kits for detecting the presence of HIV-1 in a biological sample. For example, the kit can comprise: a labeled compound or agent capable of detecting HIV-1 (e.g., an anti-HIV-1 scFv or monoclonal antibody) in a biological sample; means for determining the amount of HIV-1 in the sample; and means for comparing the amount of HIV-1 in the sample with a standard. The compound or agent can be packaged in a suitable container. The kit can further comprise instructions for using the kit to detect HIV-1 in a sample.

### Passive Immunization

Passive immunization has proven to be an effective and safe strategy for the prevention and treatment of viral diseases. (See Keller et al, Clin. Microbiol. Rev. 13:602-14 (2000); Casadevall, Nat. Biotechnol. 20:114 (2002); Shibata et al, Nat. Med. 5:204-10 (1999); and Igarashi et al, Nat. Med. 5:211-16 (1999), each of which are incorporated herein by reference)). Passive immunization using neutralizing human monoclonal antibodies could provide an immediate treatment strategy for emergency prophylaxis and treatment of HIV-1 while the alternative and more time-consuming development of vaccines and new drugs in underway. Previous investigations have indicated that passively administered neutralizing antibodies can confer protection against HIV-1 infection (see Parren et al, AIDS 9:F1-F6 (1995); Shibat et al, Nat. Med. 5:204-10 (1995); Gausuin et al, Nat. Med. 12:1389-93 (1997); Mascola et al, Nat. Med. 6:207-10 (2000); Trkola et al, Nat. Med. 11:615-22 (2005)). These observations suggest that passive immunization with human monoclonal antibodies could be developed for the treatment of HIV-1 (See Holmes, J. Clin. Invest. 111:1605-9 (2003)).

Based on experience with other viruses, those skilled in the art will recognize that a subunit vaccine can be designed to elicit neutralizing antibodies against HIV-1. Thus, the development of neutralizing human monoclonal antibodies and subunit vaccine candidates that are based on the epitopes on HIV-1 envelope proteins will play an important role in such therapeutic methods.

Subunit vaccines potentially offer significant advantages over conventional immunogens. They avoid the safety hazards inherent in production, distribution, and delivery of conventional killed or attenuated whole-pathogen vaccines. Furthermore, they can be rationally designed to include only confirmed protective epitopes, thereby avoiding suppressive T epitopes (see Steward et al, J. Virol. 69:7668 (1995)) or immunodominant B epitopes that subvert the immune system by inducing futile, non-protective responses (e.g. “decoy” epitopes). (See Garrity et al, J. Immunol. 159:279 (1997)).

Subunit vaccines also offer potential solutions to problems including pathogen variation and hypermutability that often plague vaccine development efforts. Only epitopes from invariant, conserved regions of a pathogen's antigenic structure need be included in the subunit vaccine, thereby ensuring long-term protection for individuals and populations. Alternatively, a cocktail of peptides representing multiple variants of an antigen could be assembled, in order to mimic a range of variants of a highly mutable epitope. (See Taboga et al, J. Virol. 71:2606 (1997)). Finally, subunit vaccines are cheaper to manufacture and more stable than many other vaccine formulations.

Moreover, those skilled in the art will recognize that good correlation exists between the antibody neutralizing activity in vitro and the protection in vivo for many different viruses, challenge routes, and animal models. (See Burton, Nat. Rev. Immunol. 2:706-13 (2002); Parren et al, Adv. Immunol. 77:195-262 (2001)). The in vitro and in vivo data presented herein suggest that one or more human monoclonal antibodies of the invention (e.g., HJ16.22, HK20.26, or HGN194.12), can be further developed and tested in in vivo animal studies to determine their clinical utility as potent viral entry inhibitors for emergency prophylaxis and treatment of HIV-1.

### Antigen-Ig Chimeras in Vaccination

It has been over a decade since the first antibodies were used as scaffolds for the efficient presentation of antigenic determinants to the immune systems. (See Zanetti, Nature 355:476-77 (1992); Zaghouani et al, Proc. Natl. Acad. Sci. USA 92:631-35 (1995)). When a peptide is included as an integral part of an IgG molecule, the antigenicity and immunogenicity of the peptide epitopes are greatly enhanced as compared to the free peptide. Without being bound by any theory, such enhancement may be possibly due to the antigen-IgG chimeras longer half-life, better presentation and constrained conformation, which mimic their native structures.

Moreover, an added advantage of using an antigen-Ig chimera is that either the variable or the Fc region of the antigen-Ig chimera can be used for targeting professional antigen-presenting cells (APCs). To date, recombinant Igs have been generated in which the complementarity-determining regions (CDRs) of the heavy chain variable gene (VH) are replaced with various antigenic peptides recognized by B or T cells. Such antigen-Ig chimeras have been used to induce both humoral and cellular immune responses. (See Bona et al, Immunol. Today 19:126-33 (1998)).

Chimeras with specific epitopes engrafted into the CDR3 loop have been used to induce humoral responses to either HIV-1 gp120 V3-loop or the first extracellular domain (D1) of human CD4 receptor. (See Lanza et al, Proc. Natl. Acad. Sci. USA 90:11683-87 (1993); Zaghouani et al, Proc. Natl. Acad. Sci. USA 92:631-35 (1995)). The immune sera were able to prevent infection of CD4 SupT1 cells by HIV-1MN (anti-gp120 V3C) or inhibit syncytia formation (anti-CD4-D1). The CDR2 and CDR3 can be replaced with peptide epitopes simultaneously, and the length of peptide inserted can be up to 19 amino acids long.

Alternatively, one group has developed a “troybody” strategy in which peptide antigens are presented in the loops of the Ig constant (C) region and the variable region of the chimera can be used to target IgD on the surface of B-cells or MHC class II molecules on professional APCs including B-cells, dendritic cells (DC) and macrophages. (See Lunde et al, Biochem. Soc. Trans. 30:500-6 (2002)).

An antigen-Ig chimera can also be made by directly fusing the antigen with the Fc portion of an IgG molecule. You et al, Cancer Res. 61:3704-11 (2001) were able to obtain all arms of specific immune response, including very high levels of antibodies to hepatitis B virus core antigen using this method.

### DNA Vaccination

DNA vaccines are stable, can provide the antigen an opportunity to be naturally processed, and can induce a longer-lasting response. Although a very attractive immunization strategy, DNA vaccines often have very limited potency to induce immune responses. Poor uptake of injected DNA by professional APCs, such as dendritic cells (DCs), may be the main cause of such limitation. Combined with the antigen-Ig chimera vaccines, a promising new DNA vaccine strategy based on the enhancement of APC antigen presentation has been reported (see Casares, et al, Viral Immunol. 10:129-36 (1997); Gerloni et al, Nat. Biotech. 15:876-81 (1997); Gerloni et al, DNA Cell Biol. 16:611-25 (1997); You et al, Cancer Res. 61:3704-11 (2001)), which takes advantage of the presence of Fc receptors (FcγRs) on the surface of DCs.

It is possible to generate a DNA vaccine encoding an antigen (Ag)-Ig chimera. Upon immunization, Ag-Ig fusion proteins will be expressed and secreted by the cells taking up the DNA molecules. The secreted Ag-Ig fusion proteins, while inducing B-cell responses, can be captured and internalized by interaction of the Fc fragment with FcγRs on DC surface, which will promote efficient antigen presentation and greatly enhance antigen-specific immune responses. Applying the same principle, DNA encoding antigen-Ig chimeras carrying a functional anti-MHC II specific scFv region gene can also target the immunogens to all three types of APCs. The immune responses could be further boosted with use of the same protein antigens generated in vitro (i.e., “prime and boost”), if necessary. Using this strategy, specific cellular and humoral immune responses against infection of influenza virus were accomplished through intramuscular (i.m.) injection of a DNA vaccine. (Casares et al, Viral. Immunol. 10:129-36 (1997)).

### Vaccine Compositions

Therapeutic or prophylactic compositions are provided herein, which generally comprise mixtures of one or more monoclonal antibodies or ScFvs and combinations thereof. The prophylactic vaccines can be used to prevent HIV-1 infection and the therapeutic vaccines can be used to treat individuals following HIV-1 infection. Prophylactic uses include the provision of increased antibody titer to HIV-1 in a vaccination subject. In this manner, subjects at high risk of contracting HIV-1 can be provided with passive immunity to HIV-1.

These vaccine compositions can be administered in conjunction with ancillary immunoregulatory agents. For example, cytokines, lymphokines, and chemokines, including, but not limited to, IL-2, modified IL-2 (Cys125 to Ser125), GM-CSF, IL-12, γ-interferon, IP-10, MIP1β, and RANTES.

### Evaluation of Antigenic Protein Fragments (APFs) for Vaccine Potential

A vaccine candidate targeting humoral immunity must fulfill at least three criteria to be successful: it must provoke a strong antibody response (“immunogenicity”); a significant fraction of the antibodies it provokes must cross-react with the pathogen (“immunogenic fitness”); and the antibodies it provokes must be protective. While immunogenicity can often be enhanced using adjuvants or carriers, immunogenic fitness and the ability to induce protection (as evidenced by neutralization) are intrinsic properties of an antigen which will ultimately determine the success of that antigen as a vaccine component.

### Evaluation of Immunogenic Fitness

“Immunogenic fitness” is defined as the fraction of antibodies induced by an antigen that cross-react with the pathogen. (See Matthews et al, J. Immunol. 169:837 (2002)). It is distinct from immunogenicity, which is gauged by the titer of all of the antibodies induced by an antigen, including those antibodies that do not cross-react with the pathogen. Inadequate immunogenic fitness has probably contributed to the disappointing track record of peptide vaccines to date. Peptides that bind with high affinity to antibodies and provoke high antibody titers frequently lack adequate immunogenic fitness, and, therefore, they fail as potential vaccine components. Therefore, it is important to include immunogenic fitness as one of the criteria for selecting HIV-1 vaccine candidates.

A common explanation for poor immunogenic fitness is the conformational flexibility of most short peptides. Specifically, a flexible peptide may bind well to antibodies from patients, and elicit substantial antibody titers in naive subjects. However, if the peptide has a large repertoire of conformations, a preponderance of the antibodies it induces in naive subjects may fail to cross-react with the corresponding native epitope on intact pathogen.

Like short peptides, some APFs may be highly flexible and, therefore may fail as vaccine components. The most immunogenically fit APFs are likely to consist of self-folding protein subdomains that are intrinsically constrained outside the context of the whole protein.

Because immunogenic fitness is primarily a property of the APF itself, and not of the responding immune system, immunogenic fitness can be evaluated in an animal model (e.g. in mice) even though ultimately the APF will have to perform in humans.

The immunogenic fitness achieved by APFs is evaluated by immunosorption of anti-APF sera with polyproteins, in a procedure analogous to that described in Matthews et al, J. Immunol. 169:837 (2002). IgG is purified from sera collected from mice that have been immunized. Purified, biotinylated polyproteins are mixed with the mouse IgG and incubated. Streptavidin-coated sepharose beads are then added in sufficient quantity to capture all of the biotinylated polyproteins, along with any bound IgG. The streptavidin-coated beads are removed by centrifugation at 13,000 rpm in a microcentrifuge, leaving IgG that has been depleted of antibodies directed against the polyproteins. Mock immunosorptions are performed in parallel in the same way, except that biotinylated BSA will be substituted for polyproteins as a mock absorbent.

To measure the immunogenic fitness of APFs, the polyprotein-absorbed antibodies and the mock-absorbed antibodies are titered side-by-side in ELISA against the immunizing APF. For APFs affinity selected from a phage display NPL, the antigen for these ELISAs will be purified APF-GST fusion proteins. For the potentially glycosylated APFs from the mammalian cell display NPL, the antigen for these ELISAs will be APF-Fc fusion proteins secreted by mammalian cells and purified with protein A. The percentage decrease in the anti-APF titer of polyprotein-absorbed antibodies compared with the mock-absorbed antibodies will provide a measure of the immunogenic fitness of the APF.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x+10%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis and therapy. The terms “subject” or “patient” are used interchangeably herein to mean all mammals including humans. Examples of subjects include humans, cows, dogs, cats, horses, goats, sheep, pigs, and rabbits. In one embodiment, the patient is a human.

### EXAMPLES

Exemplary embodiments of the present invention are provided in the following examples. The following examples are presented only by way of illustration and to assist one of ordinary skill in using the invention. The examples are not intended in any way to limit the scope of the invention.

### Example 1

### Isolation of Human Anti-HIV-1 Mabs

Twenty one HIV-1-infected donors were selected on the basis of the clade of their infecting virus (predominantly non-B clade) and on the ability of their plasma to neutralize a panel of HIV-1 primary isolates using PBMC- or TZMbl-based assay. IgG+ memory B cells were isolated from PBMC of these donors and immortalized using a previously described improved EBV immortalization method (Traggiai E, et al. (2004) Nat Med 10: 871-875). The immortalization efficiency of memory B cells from HIV-1 patients was significantly lower than that of non-HIV-1 infected donors (3% versus 20%, n=21, p<0.001). This finding may be explained by the recent report that HIV-specific B cells are present within a population of “exhausted” memory B cells, characterized by the expression of inhibitory receptors and low levels of CD21 (Moir S, Fauci A S (2009) Nat Rev Immunol. 9: 235-245; Moir S, et al. (2008) J Exp Med 205: 1797-1805).

Culture supernatants were harvested 14 days after immortalization and assayed in parallel for binding to five recombinant HIV-1 Env proteins: trimeric gp140 proteins (UG37, clade A and CN54, clade C), monomeric gp120 (CN54, CRF07_BC and IIIB, clade B) and gp41 recombinant ectodomain (HxB2, clade B). Briefly, ELISA plates were coated with Env antigens, blocked with 10% FCS in PBS, incubated with human mAbs and washed. Bound mAbs were detected by incubation with AP-conjugated goat anti-human IgG (Southern Biotech). Plates were then washed, substrate (p-NPP, Sigma) was added and plates were read at 405 nm. The relative affinities of mAbs binding to respective coated antigens were determined by measuring the concentration of each mAb required to achieve 50% maximal binding at saturation (K50).

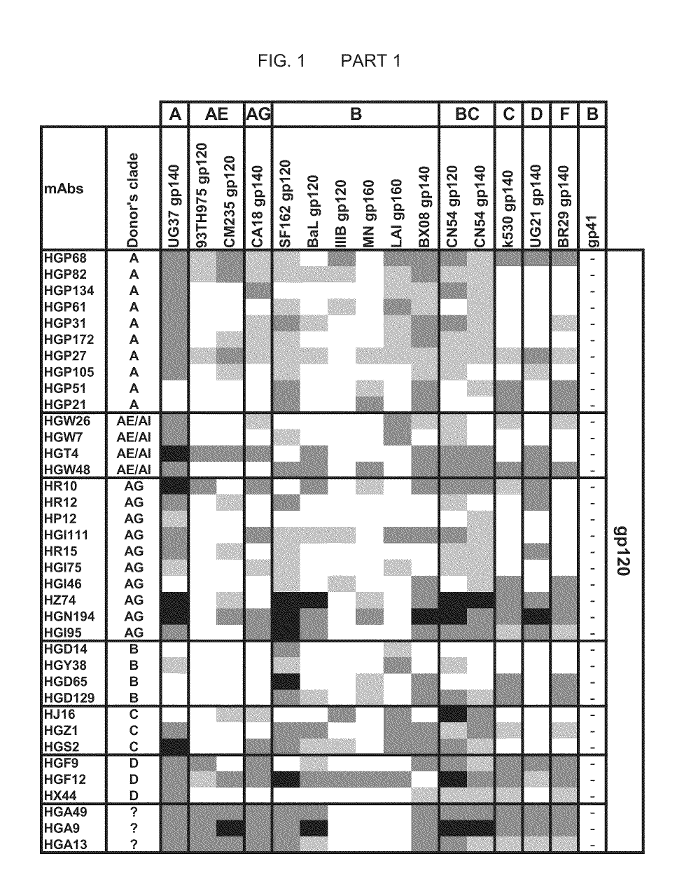

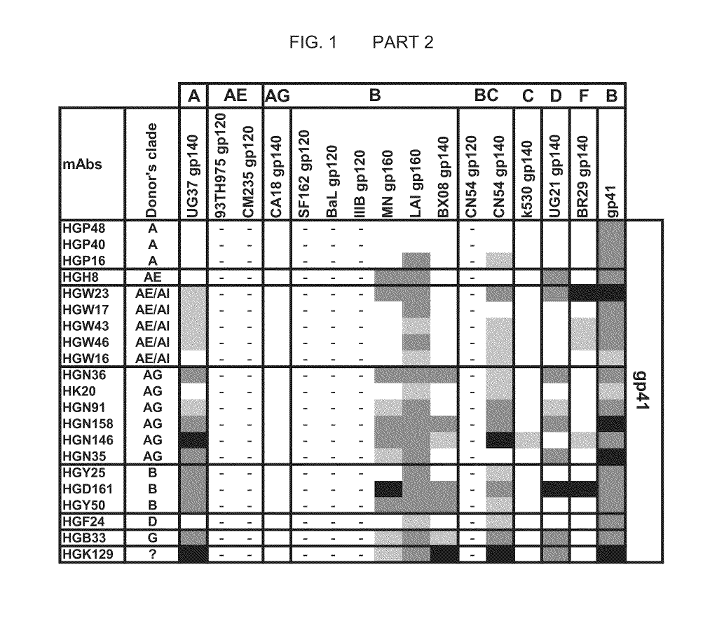

From the 21 donors interrogated we selected 58 B cell clones producing mAbs that bound to at least one of the screening antigens. The mAbs were purified and further characterized for binding specificity using an extended panel of recombinant Env proteins representative of several HIV-1 clades with diverse coreceptor usage, geographic origin and conformation. The binding data for each mAb are displayed in FIG. 1 and expressed as half-maximal binding concentrations at equilibrium (K50), which correlate with the equilibrium dissociation constants (Zhang M Y, et al. (2004) J Virol 78: 9233-9242). Of the 58 mAbs, 37 bound to gp120 and 21 to gp41. Several gp120-specific and gp41-specific mAbs showed a broad pattern of reactivity with most recombinant proteins, although usually within a broad range of K50 values. Overall, there was no relationship between the donor's HIV-1 clade and the clade specificity of the isolated mAbs.

### Example 2

### Neutralization Activity of Human Anti-HIV-1 Mabs

To measure the neutralization activity of the isolated mAbs, luciferase-encoding virions pseudotyped with the desired HIV-1 Env proteins were used in a single-cycle infectivity assay, as previously described (Gorny M K, et al. (2002) J Virol 76: 9035-9045). Briefly, appropriate dilutions of the virion-containing culture supernatants were pre-incubated at 37° C. for 1 h with mAbs at various concentrations. The virus-mAb mixtures were added to HOS-CD4-CCR5 cells and incubated for 3 days at 37° C. A similar protocol was used for supernatants screening using TZM-bl cells (Li M, et al. (2006) J Virol 80: 11776-11790). The cells were then lysed with Britelite reagent (Perkin-Elmer) and the relative light units in the cell lysates were determined on a luminometer microplate reader (Veritas, Turner Biosystems). The 50% inhibitory dose (IC50) was defined as the sample concentration at which relative luminescence units were reduced 50% compared to virus control wells.

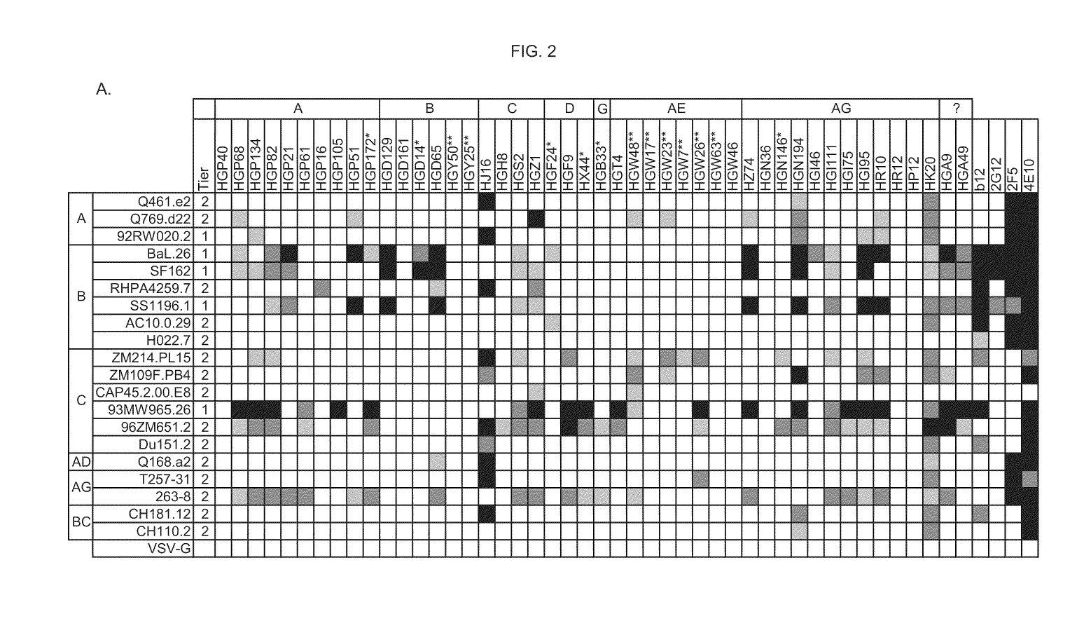

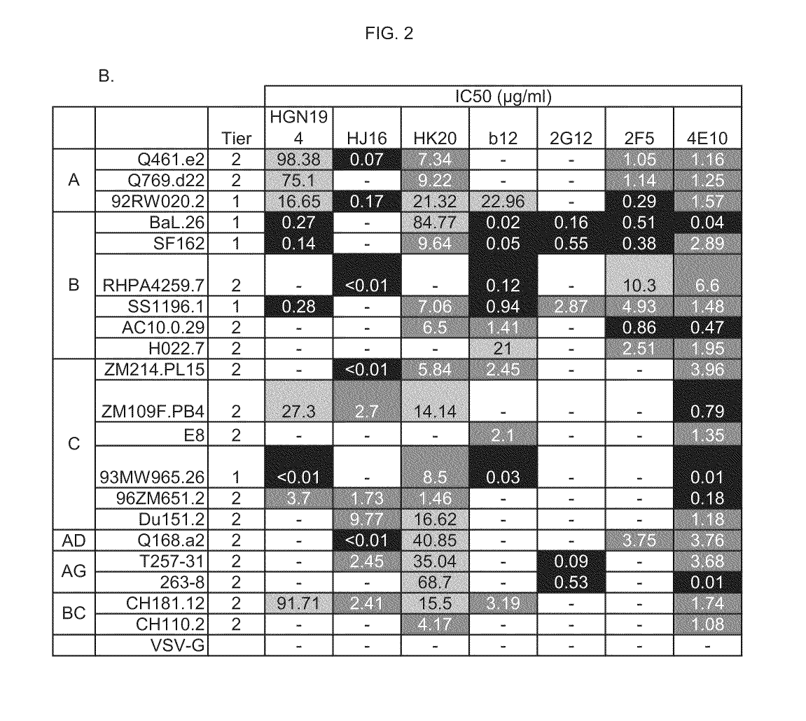

The neutralizing activity of the mAbs was measured using 20 pseudotyped HIV-1 primary isolate envs characterized by different sensitivity to neutralization (Li M, et al. (2005) J Virol 79: 10108-10125; Li M, et al. (2006) J Virol 80: 11776-11790). The mAbs were tested at a fixed concentration (100 μg/ml) in the luciferase based neutralization assay using HOS-CD4.R5 as target cells. Out of the 46 mAbs tested against the whole virus panel, 37 showed neutralizing activity on at least one isolate (FIG. 2A). Three mAbs, HJ16, HGN194 and HK20, stood out for their breadth of neutralizing activity, neutralizing 10, 11 and 17 out of the 20 pseudoviruses, respectively (see FIG. 2A). These mAbs were further characterized in the same assay for their potency (see FIG. 2B). HJ16 showed high neutralizing activity, while HGN194 and HK20 were less potent. In addition, while most antibodies preferentially neutralized Tier-1 isolates, HJ16 preferentially neutralized Tier-2 isolates (FIGS. 2A-B).

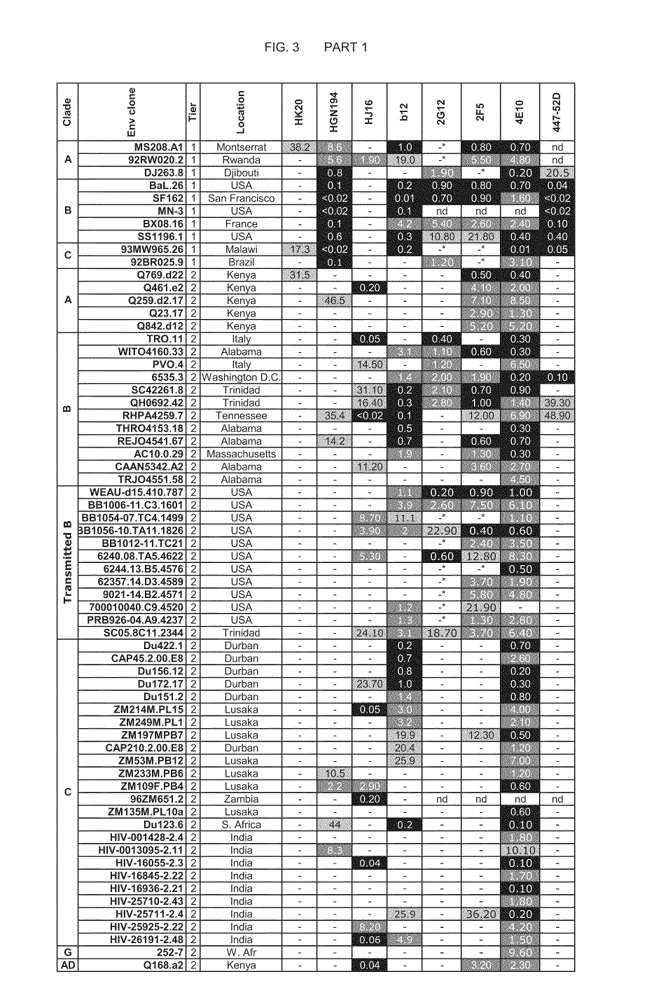

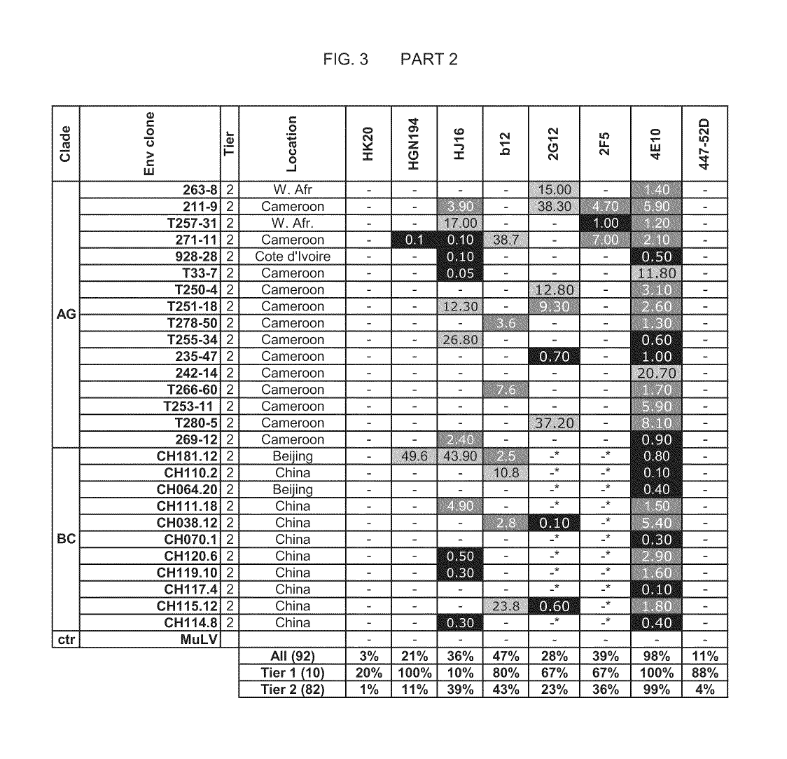

Since it has been reported that HIV-1 neutralization is target cell type sensitive (Fenyo E M, et al. (2009) the NeutNet report. PLoS ONE 4: e4505; Choudhry V, et al. (2006) Biochem Biophys Res Commun 348: 1107-1115), we used TZM-bl as target cells and titrated the mAbs against a larger panel (n=92) of predominantly Tier-2 pseudoviruses (FIG. 3).

We established the breadth of neutralization of these three novel mAbs, by comparing them in the TZM-bl assay with the five mAbs currently considered to be the most broadly reactive, namely b12, 2G12, 2F5, 4E10 and 447-52D. We used a large multi-clade panel comprising 10 Tier-1 and 82 Tier-2 isolates including clade B early-transmitted viruses (Keele B F, et al. (2008) Proc Natl Acad Sci USA 105: 7552-7557). HJ16, HGN194 and HK20 showed a pattern that was clearly distinct from that of previously described mAbs. In particular, HGN194 neutralized with high potency all Tier-1 viruses from clade A, B and C and 11% of Tier-2 viruses. By contrast HJ16 neutralized only 1 out of 10 Tier-1 viruses, but neutralized 39% of Tier-2 viruses. Thus HJ16 is comparable to b12 and 2F5 in terms of percentage of Tier-2 isolate neutralization and is superior to 2G12 and 447-52D. 4E10, as previously reported, showed an extremely broad pattern of reactivity. Taken together the above results indicate that neutralizing mAbs can be isolated from memory B cells of HIV-1 infected individuals, some of which display broad neutralizing activity.

### Example 3

### Epitope Mapping of Anti-HIV-1 Human Mabs

To define the epitopes to which the anti-HIV-1 human mAbs bind two different approaches were used: scanning of peptide libraries (Pepscan analysis), cross-competition assays and sCD4 binding inhibition.

Overlapping linear 15-mer and cyclized 15-mer peptides based on gp160 of HIV-1 UG037 and 93MW965 and the overlapping 15-mer peptides of gp41 strain SF162 were synthesized on polypropylene support (minicards), and were tested for reactivity with mAbs as described (Langedijk J P, et al. (1997)J Virol 71: 4055-4061; Timmerman P, et al. (2005) Chembiochem 6: 821-824).

For competition assays mAbs were purified on Protein G columns (GE Healthcare) and biotinylated using the EZ-Link NHS-PEO solid phase biotinylation kit (Pierce). The competition between unlabeled and biotinylated mAbs for binding to immobilized Env antigens was measured by ELISA. Briefly, unlabelled competitor mAbs were added at different concentrations. After 1 hour biotinylated mAbs were added at a concentration corresponding to the 70-80% of the maximal OD level. After incubation for 1 h, plates were washed and bound biotinylated mAb was detected using AP-labeled streptavidin (Jackson Immunoresearch). The percentage of inhibition was tested in triplicates and calculated as follow: (1−[(ODsample−ODneg ctr)/(ODpos ctr−ODneg ctr)])×100.

The ability of mAbs to inhibit binding of sCD4 to gp120 or gp140 was evaluated by ELISA. Serial dilutions of mAbs were pre-incubated with gp120 (or gp140) and added to plates pre-coated with sCD4. After 1 h plates were washed and incubated with sheep polyclonal antibody D7324 (Aalto Bio-Reagents) followed by washing, incubation with AP-conjugated rabbit anti-sheep IgG antibody (Abcam, Cambridge, UK), extensive washing and detection using p-NPP (Sigma) as substrate.

### Example 4

### HJ16, a Neutralizing mAb Binding a Novel Epitope Proximal to the CD4Bs

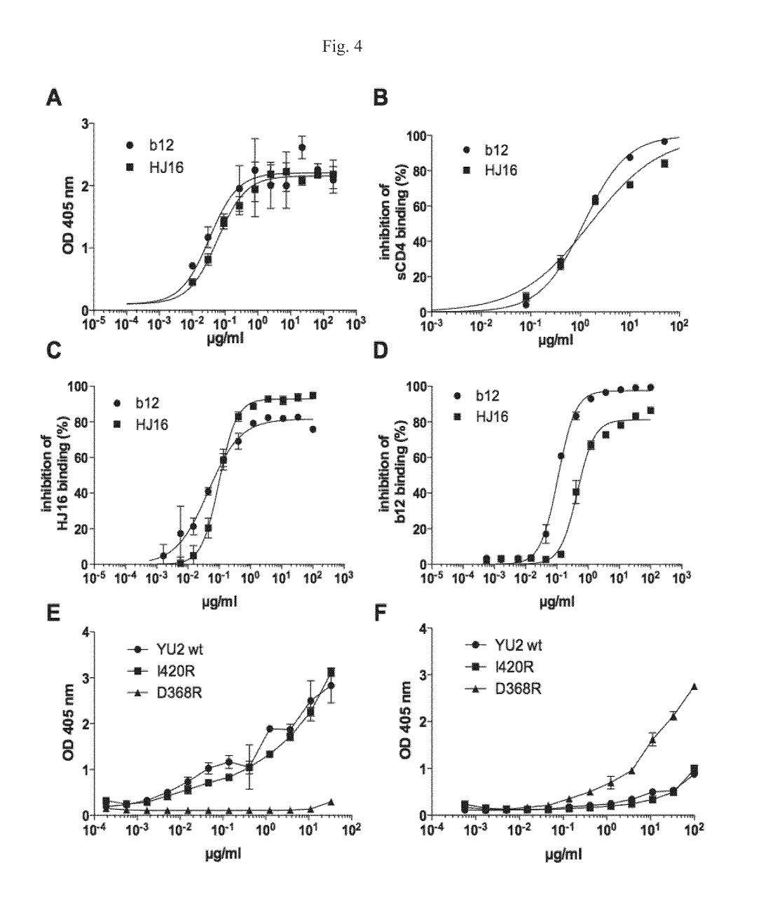

MAb HJ16, derived from a donor infected with clade C, has a unique neutralization profile with potent and selective neutralization of multiple Tier-2 pseudoviruses (FIG. 2B and FIG. 3). When compared with the CD4bs-specific mAb b12 for gp120 binding, HJ16 showed similar binding characteristics (FIG. 4A) and inhibited to a comparable extent the binding of gp120 to solid-phase sCD4 (FIG. 4B, IC50 values of 1.57 and 1.16 μg/ml, respectively). However, cross-competition analysis of HJ16 and b12 for binding to gp120 revealed incomplete heterologous inhibition, with plateau values of approximately 80% (FIGS. 4C-D). These results suggest that HJ16 and b12 recognize close but non-overlapping CD4bs-proximal epitopes. To further characterize HJ16 specificity we measured its binding to two YU2 gp120 mutants: the D368R mutant, which is not bound by CD4 or CD4bs-specific mAbs (Li Y, et al. (2007) Nat Med 13: 1032-1034) and the I420R mutant, which is not recognized by CD41-specific mAbs (Scheid J F, et al. (2009) Nature 458: 636-640). As already reported, b12 bound the I420R CD41 mutant, but failed to recognize the D368R CD4bs mutant. By contrast, HJ16 bound both mutants and indeed bound better to the D368R CD4bs mutant compared to the wild-type molecule (FIGS. 4E-F). Taken together these results are consistent with HJ16 recognition of an epitope close to, but distinct from, that recognized by b12. Attempts to map the epitope by Pepscan analysis using overlapping linear peptides were unsuccessful (not shown), suggesting that HJ16 recognizes a discontinuous epitope. HJ16 and b12 neutralized 1/10 and 8/10 Tier-1 and 32/82 and 35/82 Tier-2 pseudoviruses, respectively (FIG. 3). Interestingly, 22/32 Tier-2 isolates neutralized by HJ16 were not neutralized by b12, and reciprocally 24/35 Tier-2 isolates neutralized by b12 were not neutralized by HJ16. Another interesting finding is that HJ16 does not discriminate between clades as much as b12, 2G12 and 2F5 (e.g. b12 and 2G12 rarely neutralize clade A isolates while 2F5 and 2G12 rarely neutralize clade C isolates, Table 3).

TABLE 3
Percentage of HIV-1 isolates neutralized in the TZM-bl based neutralization
assay shown in FIG. 3

| | | HK20 | HGN194 | HJ16 | b12 | 2G12 | 2F5 | 4E10 | 447-52D |
|---|---|---|---|---|---|---|---|---|---|
| | All (92) | 3% | 21% | 36% | 47% | 28% | 39% | 98% | 11% |
| | Tier 1 (10) | 20% | 100% | 10% | 80% | 67% | 67% | 100% | 88% |
| | Tier 2 (82) | 1% | 11% | 39% | 43% | 23% | 36% | 99% | 4% |
| Tier 1 + Tier 2 | Clade A (8) | 25% | 38% | 25% | 25% | 13% | 88% | 100% | 0% |
| | Clade AD (1) | 0% | 0% | 100% | 0% | 0% | 100% | 100% | 0% |
| | Clade AG (16) | 0% | 0% | 50% | 25% | 38% | 25% | 100% | 0% |
| | Clade B (29) | 0% | 24% | 34% | 69% | 55% | 79% | 97% | 28% |
| | Clade BC (11) | 0% | 0% | 45% | 36% | 18% | 0% | 100% | 0% |
| | Clade C (26) | 4% | 23% | 27% | 54% | 0% | 8% | 100% | 4% |
| | Clade G (1) | 0% | 0% | 0% | 0% | 0% | 0% | 100% | 0% |
| Tier 2 | Clade A (5) | 14% | 20% | 20% | 0% | 0% | 100% | 100% | 0% |
| | Clade AD (1) | 0% | 0% | 100% | 0% | 0% | 100% | 100% | 0% |
| | Clade AG (16) | 0% | 0% | 50% | 25% | 38% | 25% | 100% | 0% |
| | Clade B (24) | 0% | 8% | 42% | 63% | 50% | 75% | 97% | 13% |
| | Clade BC (11) | 0% | 0% | 45% | 36% | 18% | 0% | 100% | 0% |
| | Clade C (24) | 0% | 17% | 29% | 54% | 0% | 9% | 100% | 0% |
| | Clade G (1) | 0% | 0% | 0% | 0% | 0% | 0% | 100% | 0% |

Shown is the percentage of HIV-1 isolates neutralized by each mAb as indicated in FIG. 3.
In parenthesis are the number of isolates in each analyzed group.

These results reveal a largely non-overlapping pattern of reactivity of HJ16 and b12 and suggest that the combination of these two mAbs could be effective against a dominant fraction of HIV-1 isolates (57/82, i.e. 69%).

### Example 5

### HGN194, a Broadly Neutralizing mAb that Binds to the V3 Crown

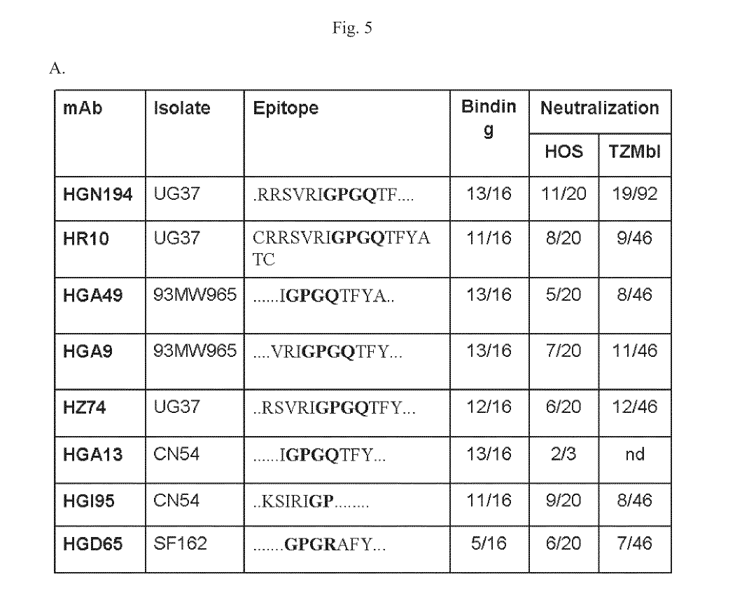

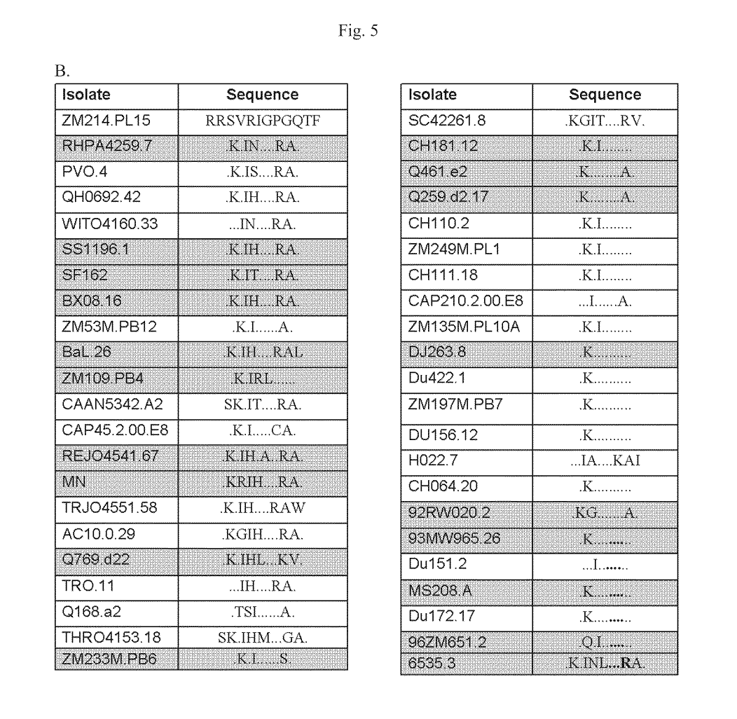

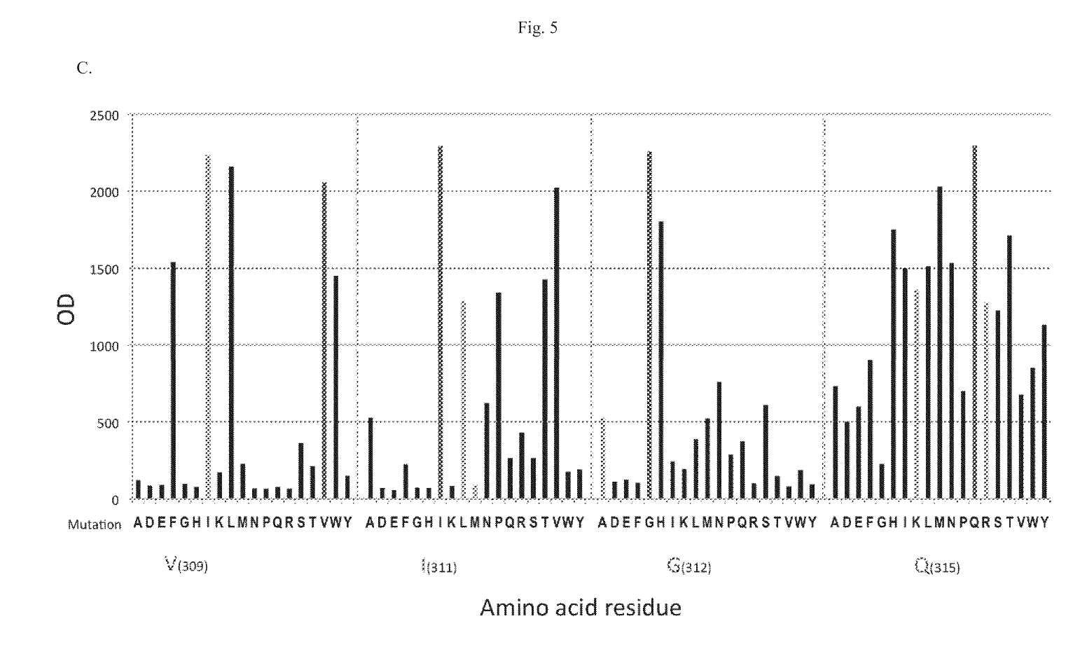

MAb HGN194, isolated from a donor infected with CRF02_AG clade, neutralized 11/20 pseudoviruses in the HOS-based assay and 19/92 pseudoviruses in the TZM-blbased assay. Of note, HGN194 neutralized all Tier-1 isolates tested (10/10 neutralized). Using Pepscan analysis with linear and cyclic peptide libraries of gp120 the epitope recognized by HGN194 was mapped to the sequence RRSVRIGPGQTF (SEQ ID NO: 49) in the crown of the V3-loop (FIG. 5A). Similarly, the epitope of 7 additional gp120-specific mAbs was mapped to the same V3 region using different peptide libraries generated from the sequence of the isolate to which each mAb was mostly reactive (FIG. 5A). The minimal sequence recognized by these mAbs ranged from 7 to 17 amino acids and, with a single exception, comprising the consensus sequence G(A)PGR/Q/K, which interacts with CCR5 or CXCR4 during the viral entry process. The consensus sequence represents the four possible sequences found in the analyzed isolates (see FIG. 5B)—GPGR (SEQ ID NO: 50), GPGQ (SEQ ID NO: 51), GPGK (SEQ ID NO: 52) and APGR (SEQ ID NO: 53). However, in contrast to HGN194 which neutralizes with high potency all Tier-lisolates tested, the other V3-specific mAbs neutralized only a few Tier-1 isolates (FIGS. 2A-B and FIG. 3).

To better characterize the epitope recognized by HGN194 we performed a replacement scanning of each position with the 18 complementary amino acids. This analysis revealed only 3 positions (i.e. RRSVRIGPGQTF) where amino acid substitutions abrogated binding. Remarkably, only one mutation out of the 21 found in viral isolates (I to M in position 6) affected binding of HGN194 (FIGS. 5B-C). However, we observed that several HIV-1 isolates that were not neutralized by HGN194 encoded the same amino acidic sequence shared by other HIV-1 isolates that were neutralized (FIG. 5B). For instance, the epitope RKSVRIGPGQTF (SEQ ID NO: 54) on 93MW965.26, which is strongly neutralized by HGN194 (i.e. IC50 <0.02 μg/ml), is shared with Du422.1, ZM197M.PB7 and Du172.17, being these isolates not neutralized by HGN194.

Taken together these results indicate that HGN194 is unique among V3-specific antibodies both for its potency and breadth. Of note, HGN194 appears to have a broader reactivity than 447-52D since it neutralizes all Tier-1 isolates and 11% of Tier-2 isolates, while 447-52D neutralizes 88% of Tier-1 and 4% of Tier-2 isolates (FIG. 3 and Table 3).

### Example 6

### HK20, an HR-1 Specific mAb with Target Cell-Specific Neutralizing Activity

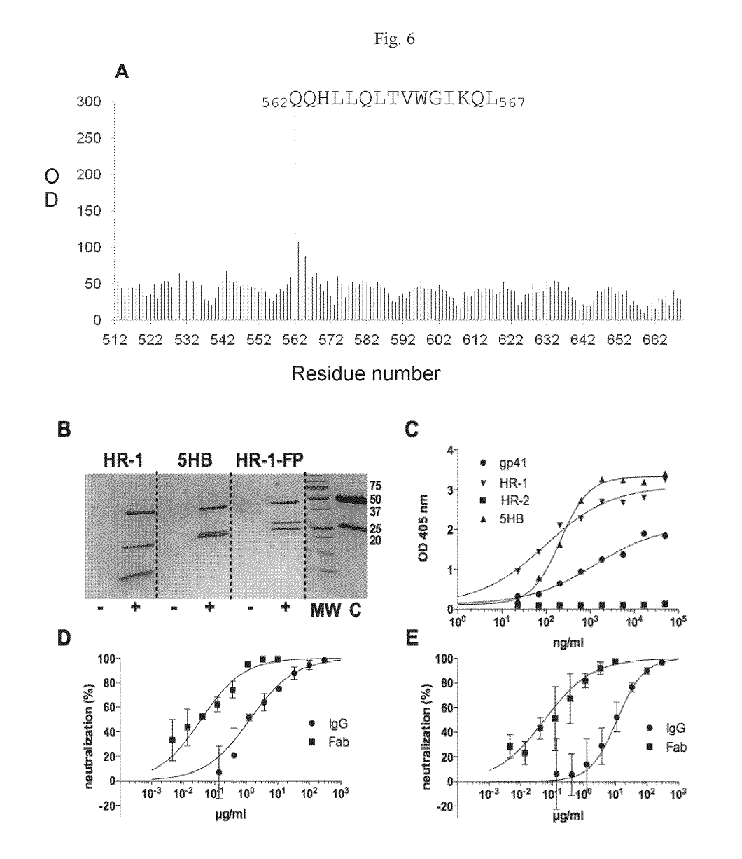

HK20 was initially characterized as a gp41-specific mAb with broad neutralizing activity in the HOS-based assay but lacking activity in the TZM-bl assay. To map the epitope, we tested HK20 against all overlapping 15-mer peptides of the extracellular region of HIV-1 gp41. HK20 bound peptide QQHLLQLTVWGIKQL (SEQ ID NO: 55) that overlaps the hydrophobic pocket sequence of HR-1 (FIG. 6A). The specificity of HK20 was confirmed by immunoprecipitation of the 5-helix bundle (5HB) construct (Root M J, et al. (2001) Science 291: 884-888) and of a trimeric HR-1 construct that includes the gp41 fusion peptide, indicating that the fusion peptide does not interfere with HK20 binding (FIG. 6B). In ELISA, HK20 bound to 5HB and HR-1 gp41 constructs with K50 values of 210 and 95 ng/ml, respectively and also to the gp41 ectodomain, although with lower avidity (1.27 μg/ml) (FIG. 6C), a finding that may be due to the partial unfolding of solid phasebound gp41. Taken together, the above results indicate that HK20 recognizes a highly conserved site within the HR-1 region of gp41. Since HR-1 occupies a restricted surface only transiently exposed during the HIV-1 entry process, we hypothesized that accessibility of the HK20 target epitope might be limited by the size of an IgG molecule. We therefore compared intact IgG and Fab fragments of HK20 for their capacity to neutralize a panel of 22 pseudoviruses in the HOS-based assay. The HK20 Fab fragments showed markedly increased breadth and potency compared to IgG, being able to neutralize 21/22 isolates with IC50 values ranging from 0.01 to 5.7 μg/ml (FIGS. 6D-E and Table 4).

TABLE 4
HK20 Fab fragment shows increase in neutralization
breadth and potency

| | |
|---|---|
| IgG | Fab |

IC50 (μg/ml)

| HIV-1 isolate | Clade | IgG | Fab |
|---|---|---|---|
| Q461.e2 | A | 7.34 | 0.49 |
| Q769.d22 | A | 9.22 | 0.11 |
| Q168.a2 | AD | 40.85 | 0.12 |
| T257-31 | AG | 35.04 | 0.16 |
| 263-8 | AG | 68.70 | 1.19 |
| SF162 | B | 9.64 | 0.27 |
| SC42261.8 | B | 10.03 | 0.15 |
| CAAN5342.A2 | B | 9.63 | 0.09 |
| JRFL | B | >100 | >10 |
| BaL | B | 84.77 | 0.28 |
| THRO4153.67 | B | >100 | 0.48 |
| H022.7 | B | >100 | 5.70 |
| CH181.12 | BC | 15.50 | 0.43 |
| CH064.20 | BC | 11.10 | 0.06 |
| ZM214M.PL15 | C | 5.84 | 0.01 |
| ZM53M.PB12 | C | 15.44 | 0.60 |
| ZM109F.PB4 | C | 14.14 | 0.15 |
| CAP210.2.00.E8 | C | 11.89 | 0.16 |
| Du151..2 | C | 16.62 | 0.61 |
| ZM249.PL1 | C | 6.68 | 0.25 |
| 93MW965.26 | C | 8.50 | 0.08 |
| 96ZM651.2 | C | 1.46 | 0.04 |
| VSV-G | | >100 | >10 |

HK20 IgG and Fab fragments were tested against a panel of 28 HIV-1 primary isolates in a HOS-based neutralization assay. Shown is the IC50 value in μg/ml.

By contrast, HK20 IgG neutralized 19/22 isolates with IC50 values ranging from 1.46 to 84 μg/ml. These findings suggest that the reduced size of the Fab molecule allows increased access to the HR-1 region and imply that HR-1 accessibility is largely dependent on the target cell used.

We then tested whether further reducing the size of HK20 to single chain Fv (scFv) would increase neutralizing activity in the most demanding TZM-bl assay. HK20 IgG, Fab and scFv were compared in a TZM-bl-based assay against a panel of 45 Tier 1, 2 and 3 HIV-1 pseudoviruses (Table 5). The HK20 Fab showed high breadth and potency, since it neutralized 43 out of the 45 viruses with IC50 values ranging from 14 to 1000 nM. HK20 scFv showed on average a 2-6 times higher potency as compared to the Fab and neutralized all 45 pseudoviruses with IC50 values ranging from 6 to 737 nM. Interestingly, HK20 scFv neutralized clade C isolates more potently than clade B viruses. This observation was further supported by testing HK20 scFv against a larger panel of clade B and C isolates (27 and 25 isolates, respectively) (Table 6).

TABLE 5
Size dependent neutralization breadth and potency of
HK20 in the TZMbl based neutralization assay.

| | | IC50 (μg/ml) | | | IC50 (nM) | | |
|---|---|---|---|---|---|---|---|
| Clade | Env clone | IgG | Fab | scFv | IgG | Fab | scFv |
| A | Q461.e2 | — | 18.6 | 4 | — | 372 | 143 |
| | Q769.d22 | — | 31.7 | 5 | — | 634 | 177 |
| | MS208.A1 | 150 | 4 | 2 | 1000 | 80 | 84 |
| | 92RW020.2 | — | 16.8 | 5 | — | 336 | 183 |
| | Q259.d2.17 | — | 42.8 | 13 | — | 856 | 474 |
| B | SF162 | — | 29.5 | 3 | — | 590 | 109 |
| | BaL.26 | — | 22.7 | 6 | — | 454 | 214 |
| | SS1196.1 | — | 28.1 | 12 | — | 562 | 431 |
| | AC10.0.29 | — | 20.3 | 3 | — | 406 | 113 |
| | RHPA4259.7 | — | 19.9 | 11 | — | 398 | 406 |
| | TRO.11 | — | 15.8 | 6 | — | 316 | 221 |
| | MN-3 | 19 | 0.7 | 0 | 127 | 14 | 6 |
| | WITO4160.33 | — | 12 | 2 | — | 240 | 84 |
| | PVO.4 | — | 36.6 | 20 | — | 732 | 737 |
| | 6535.3 | — | — | 4 | — | — | 133 |
| | SC42261.8 | — | 9.2 | 3 | — | 184 | 120 |
| | QH0692.42 | — | 28.5 | 18 | — | 570 | 656 |
| | BX08.16 | — | — | 16 | — | — | 604 |
| | THRO4153.18 | — | 50 | 13 | — | 1000 | 490 |
| | REJO4541.67 | — | 18.6 | 4 | — | 372 | 159 |
| | CAAN5342.A2 | — | 24.4 | 4 | — | 488 | 137 |
| | TRJO4551.58 | — | 18.1 | 10 | — | 362 | 373 |
| C | Du151.2 | — | 13.8 | 9 | — | 276 | 317 |
| | ZM53M.PB12 | — | 4.3 | 3 | — | 86 | 99 |
| | CAP210.2.00.E8 | — | 6.7 | 2 | — | 134 | 75 |
| | 96ZM651.2 | 128 | 7.4 | 3 | 856 | 148 | 114 |
| | Du422.1 | — | 4.4 | 1 | — | 88 | 55 |
| | CAP45.2.00.E8 | — | 13.5 | 2 | — | 270 | 77 |
| | Du156.12 | 121 | 5.9 | 2 | 805 | 118 | 59 |
| | Du172.17 | 149 | 7.7 | 1 | 991 | 154 | 49 |
| | ZM214M.PL15 | — | 8 | 1 | — | 160 | 43 |
| | ZM249M.PL1 | — | 8.1 | 2 | — | 162 | 69 |
| | ZM197MPB7 | — | 21.1 | 6 | — | 422 | 236 |
| | ZM233M.PB6 | 99 | 2.9 | 1 | 660 | 58 | 41 |
| | ZM109F.PB4 | — | 10.1 | 3 | — | 202 | 122 |
| | ZM135M.PL10a | — | 10.3 | 3 | — | 206 | 112 |
| AD | Q168.a2 | — | 18.5 | 6 | — | 370 | 213 |
| AG | T257-31 | — | 20.9 | 7 | — | 418 | 273 |
| | 263-8 | — | 24.2 | 4 | — | 484 | 154 |
| | 211-9 | — | 13.8 | 3 | — | 276 | 117 |
| | 271-11 | — | 43.9 | 12 | — | 878 | 431 |
| BC | CH064.20 | 125 | 15.6 | 3 | 831 | 312 | 118 |
| | CH110.2 | — | 8.8 | 2 | — | 176 | 88 |
| | CH181.12 | — | 13.3 | 4 | — | 266 | 134 |
| | CH111.18 | — | 17.1 | 4 | — | 342 | 138 |
| | MuLV | — | — | — | — | — | — |

HK20 IgG, Fab and scFv were tested for their capacity to neutralize 45 HIV-1 pseudoviruses representing 6 different clades including Tier-1, Tier-2 and Tier-3 isolates using TZM-bl as target cells.
Shown are IC50 values expressed in μg/ml and nM. —, IC50 > 1000 nM.

TABLE 6
HK20 scFv neutralizes more potently clade C viruses.

| | | μg/ml | | nM | |
|---|---|---|---|---|---|
| Clade | Virus | IC50 | IC90 | IC50 | IC90 |
| B | SF162.LS | 3 | 37 | 109 | 1363 |
| | BaL.26 | 6 | 41 | 213 | 1502 |
| | SS1196.1 | 12 | 47 | 430 | 1727 |
| | AC10.0.29 | 3 | 22 | 113 | 831 |
| | RHPA4259.7 | 11 | >50 | 406 | — |
| | MN-3 | 0 | 2 | 6 | 64 |
| | Bx08.16 | 16 | 50 | 602 | 1852 |
| | 6535.3 | 4 | >50 | 133 | — |
| | QH0692.42 | 18 | >50 | 655 | — |
| | SC422661.8 | 3 | 23 | 119 | 836 |
| | PVO.4 | 20 | >50 | 735 | — |
| | TRO.11 | 6 | 42 | 220 | 1556 |
| | THRO4156.18 | 13 | >50 | 489 | — |
| | REJO4541.67 | 4 | >50 | 158 | — |
| | TRJO4551.58 | 10 | 45 | 372 | 1673 |
| | WITO4160.33 | 2 | 16 | 84 | 577 |
| | CAAN5342.A2 | 4 | 22 | 136 | 814 |
| | WEAU-d15.410.787 | 2 | 8 | 81 | 300 |
| | BB1006-11.C2.1601 | 31 | — | 1163 | — |
| | BB1054-07.TC4.1499 | 4 | — | 163 | — |
| | BB1056-10.TA11.1826 | 3 | 45 | 96 | 1667 |
| | BB101211.TC21 | 3 | 33 | 104 | 1222 |
| | H022.7 | 8 | — | 300 | — |
| | H030.4 | 9 | 44 | 326 | 1622 |
| | H077.31 | 2 | 43 | 78 | 1581 |
| | H035.18 | 13 | — | 463 | — |
| | H061.14 | 41 | — | 1530 | — |
| C | Du151.2 | 9 | 35 | 317 | 1285 |
| | ZM53M.PB12 | 3 | 15 | 99 | 555 |
| | CAP210.2.00.E8 | 2 | 16 | 74 | 596 |
| | 96ZM651.02 | 3 | 24 | 113 | 889 |
| | MW965.26 | 0 | 2 | 9 | 77 |
| | Du156.12 | 2 | 18 | 59 | 649 |
| | Du172.17 | 1 | 10 | 49 | 373 |
| | Du422.1 | 1 | 12 | 55 | 462 |
| | ZM197M.PB7 | 6 | 36 | 236 | 1343 |
| | ZM214M.PL15 | 1 | 26 | 43 | 950 |
| | ZM233M.PB6 | 1 | 5 | 41 | 199 |
| | ZM249M.PL1 | 2 | 15 | 69 | 572 |
| | ZM109F.PB4 | 3 | 24 | 121 | 886 |
| | ZM135M.PL10a | 3 | 13 | 112 | 497 |
| | CAP45.2.00.E8 | 2 | 43 | 77 | 1584 |
| | 16845-2.22 | 8 | 40 | 278 | 1485 |
| | 16936-2.21 | 2 | 5 | 59 | 196 |
| | 25711-2.4 | 8 | 28 | 304 | 1048 |
| | 0013095-2.11 | 1 | 3 | 19 | 122 |
| | 001428-2.42 | 3 | 20 | 115 | 744 |
| | 0921.v2.c14 | 3 | 20 | 93 | 726 |
| | 0041.v3.c18 | 1 | 6 | 44 | 226 |
| | 3873.v1.c24 | 3 | 50 | 119 | 1852 |
| | 6322.v4.c1 | 7 | 34 | 244 | 1274 |
| | 0077.v1.c16 | 0 | 10 | 15 | 385 |

Neutralization of 27 clade B and 25 clade C HIV-1 isolates in TZM-bl cells by HK20 scFv. Shown are IC50 and IC90 values expressed in μg/ml and nM. —, IC50 > 1850 nM.

We then compared HK20 scFv with the T-20 peptide (Fuzeon), a peptide inhibitor that targets the gp41 fusion machinery The comparison was done in both HOS and TZM-bl based assays using a panel of 20 isolates (Table 7). HK20 scFv neutralized the same HIV-1 isolates with IC50 values 10-100 fold lower in HOS cells as compared to TZM-bl cells (IC50 values ranging from 0.8 to 174 nM and 43 to 430 nM, respectively). T-20 neutralization was also more efficient in the HOS as compared to TZM-bl assay, with IC50 values ranging from <2.23 to 20 nM and 0.04 to 4.4 nM in TZM-bl and HOS assays, respectively. Interestingly, the HK20 epitope does not overlap with the T-20 binding site and consequently it is predicted that HK20 will be able to neutralize T-20 escape mutants, which have been shown to affect the 547-GIV-549 region (Rimsky L T, et al. (1998) J Virol 72: 986-993; Lu J, et al (2006) J Acquir Immune Defic Syndr 43: 60-64). Thus HK20 scFv represents a new tool to combat HIV-1 infection in general and specifically T-20-resistant viruses.

TABLE 7
Neutralization of 20 HIV-1 isolates in HOS and TZM-bl cells by
HK20 scFv and T20.

| | | IC50 (nM) | | | |
|---|---|---|---|---|---|
| | | HK20 scFv | | T20 | |
| Clade | Virus | HOS | TZMbl | HOS | TZMbl |
| A | Q461.e2 | 5.1 | 143.29 | 1.6 | 6.68 |
| | Q769.d22 | 2.5 | 176.7 | 1.7 | 20.04 |
| B | SF162 | 3.9 | 108.76 | 6.8 | 20.04 |
| | BaL.26 | 24.5 | 213.82 | 1.4 | 6.68 |
| | SS1196.1 | 11.2 | 430.97 | 0.9 | 4.45 |
| | AC10.0.29 | 3.9 | 113.22 | 1.2 | 6.68 |
| | RHPA4259.7 | 23 | 406.47 | 1.9 | 8.9 |
| | JR-FL | | Nd | 1.9 | nd |
| C | Du151.2 | 8.3 | 317.38 | 0.6 | <2.23 |
| | ZM53M.PB12 | 3.8 | 99.48 | 6.1 | <2.23 |
| | CAP210.2.00.E8 | 3.1 | 74.61 | 6 | 13.36 |
| | 96ZM651.2 | 0.8 | 113.59 | 0.6 | 8.9 |
| | MW965.26 | 2.1 | Nd | 0.6 | nd |
| | ZM214M.PL15 | 3.791 | 43.43 | 4.425 | 17.81 |
| AD | Q168.a2 | 15.5 | 213.07 | 5.7 | 8.9 |
| AG | T257-31 | 10.15 | 273.21 | 2.76 | <2.23 |
| | 263-8 | 12.8 | 153.68 | 0.04 | <2.23 |
| BC | CH064.20 | 1.9 | 118.42 | 0.7 | <2.23 |
| | CH110.2 | 7.265 | 87.61 | 0.3349 | <2.23 |
| | CH181.12 | 8.6 | 133.64 | 1.8 | <2.23 |

HK20 and T20 were tested for their capacity to neutralize the same 20 HIV-1 pseudoviruses representing 6 different clades using either HOS-based or TZM-bl as target cells. Shown are nM IC50 values.
nd, not determined.

Given the limitations inherent to pseudoviruses and cell lines we compared HK20-scFv, T-20 and Trimab (a cocktail of the broadly neutralizing mAbs Trimab, 2F5, 2G12 and b12) in a PBMC-based neutralization assays using nine primary replication competent HIV-1 isolates representative of clades A, B, C, D and E (Table 8). HK20 scFv neutralized 8 out of the 9 isolates tested with IC90 values ranging from 95 to 1667 nM. Interestingly, the two HIV-1 isolates neutralized more potently (i.e. Du174 and 92BR025) were clade C viruses. In conclusion these results demonstrate that reduction of size dramatically increased HK20 scFv breadth and potency of viral neutralization in different assays, including PBMC-based assays.

TABLE 8
Neutralization of 9 HIV-1 infectious isolates in a PBMC-based neutralization assay.

| | |
|---|---|
| IC50 (nM) | IC90 (nM) |

| | | HK20 | | | HK20 | | |
|---|---|---|---|---|---|---|---|
| Clade | Virus | scFv | Trimab | T20 | scFv | Trimab | T20 |
| A | VI 191 | 932 | 88 | 32 | 1667 | 271 | 56 |
| | 92RW009 | 252 | 152 | <16 | 956 | 290 | 44 |
| B | QH0692 | 655 | <5 | 78 | 1536 | 19 | 207 |
| | MN(P) | 108 | nd | 39 | 932 | nd | 63 |
| | J213 | >1850 | 136 | 35 | >1850 | 279 | 192 |
| C | Du174 | 30 | 46 | nd | 95 | 76 | nd |
| | 92BR025 | <29 | 19 | <16 | 184 | >333 | 29 |
| D | 92UG024 | 243 | 44 | 16 | 416 | 74 | 48 |
| E | CM244 | <111 | nd | nd | 346 | nd | nd |

HK20 scFv, Trimab (2F5, 2G12 and b12) and T-20 were tested in a PBMC-based neutralization assay against a panel of 9 HIV-1 infectious isolates from 5 different clades.
Shown are nM IC50 and IC90 values. —, IC50 > 1850 nM (50 μg/ml) in the case of HK20 scFv. —, IC50 > 333 nM (50 μg/ml) in the case of Trimab.

### Example 7

### Structure of the HK20 Fab in Complex with 5-Helix

The crystal structure of HK20-5-Helix complex was solved by molecular replacement and refined to a resolution of 2.3 Å (Sabin C, et al (2010) PLoS Pathog 6:e1001195). The variable domains (VH and VL) of the Fab approach the epitope in an ˜60° angle with respect to the 5-Helix trimer axis. HK20 employs the complementarity determining regions (CDR) H2, H3 and L3 to contact two adjacent HR1 helices. The tip of CDR H2 is a central determinant of interaction and positions Ile53 and Phe54 into a hydrophobic HR1 pocket (lined by HR1 chain A Leu565, Leu568, Thr569 and HR1 chain C Val570* and Ile573*). Furthermore CDR H2 Asp55 makes a water mediated contact to the carbonyl of Lys574 followed by a hydrophobic contact of Ile56. CDR H3 contacts HR1 chain A. The aromatic ring of Tyr97 is within π stacking distance to His564 and its orientation is supported by a hydrogen bond to the carbonyl of CDR H1 Arg31. The carbonyl of CDR H3 Ser99 hydrogen bonds to Trp571 NE1, whose aromatic ring forms a hydrophobic sandwich with the ring structure of Pro100b, itself stacked by Tyr100c. Tyr100c also participates in a network of hydrogen bonds by contacting Gln575, which makes a double dent hydrogen bond to CDR H2 Asn58. The binding contribution of the light chain is minor and restricted to hydrophobic contacts by Asp93 and Leu94 to Ala578 and Ala582 of HR1. A water molecule coordinated by CDR H3 Ser98 and the carbonyl of Tyr97 contacts HR2His 643. Docking demonstrates that three Fabs or antibodies could bind simultaneously to all three epitopes once they become exposed during the fusion reaction. The crystal structure reveals that the tip of HK20 CDR H2 occupies the hydrophobic pocket on HR1 that, in the postfusion conformation, is filled by HR2 residues Trp626, Trp631 and Ile635. In addition, the side chain of Trp571 rotates by ˜90° to accommodate binding to CDR H3. Thus the HK20 interaction blocks entry by preventing the folding of HR2 onto HR1 which is required to catalyze fusion of viral and cellular membranes in order to establish infection (Schibli D J, Weissenhorn W (2006) Mol Membr Biol 21: 361-371).

HK20 employs the similar structural principles to interact with HR1 as described for D5, a HR1-specific mAb antibody isolated from a human naïve B cell library (Miller M D, et al (2005) Proc Natl Acad Sci USA 102: 14759-14764). Both mAbs cover similar footprints on gp41, but approach their epitope with different angles, which contributes to the differences in neutralization breadth. Indeed, HK20 IgG have limited but still broader neutralization breadth than D5 IgG.

### Example 8

### Broad Coverage of gp41 and gp120 Epitopes

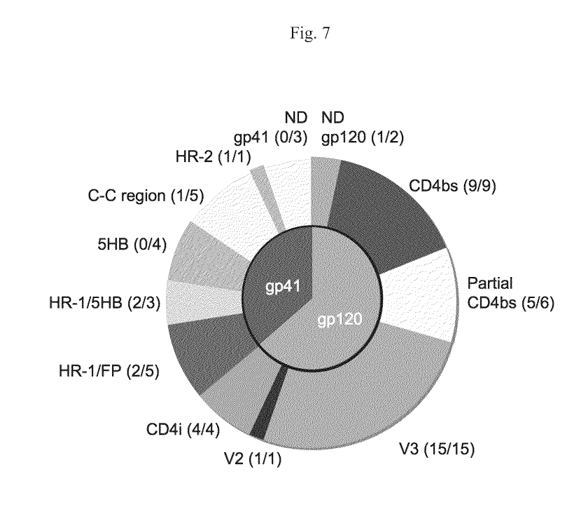

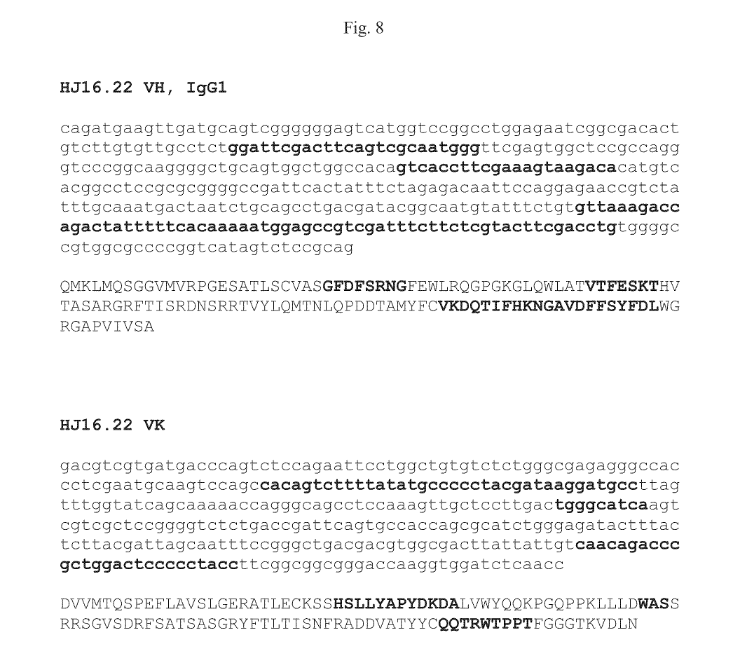

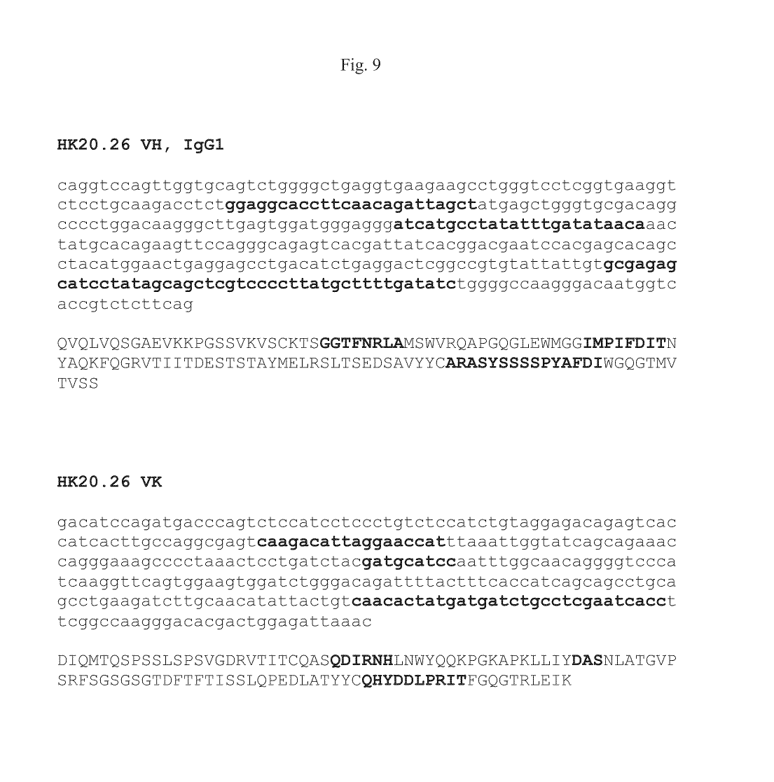

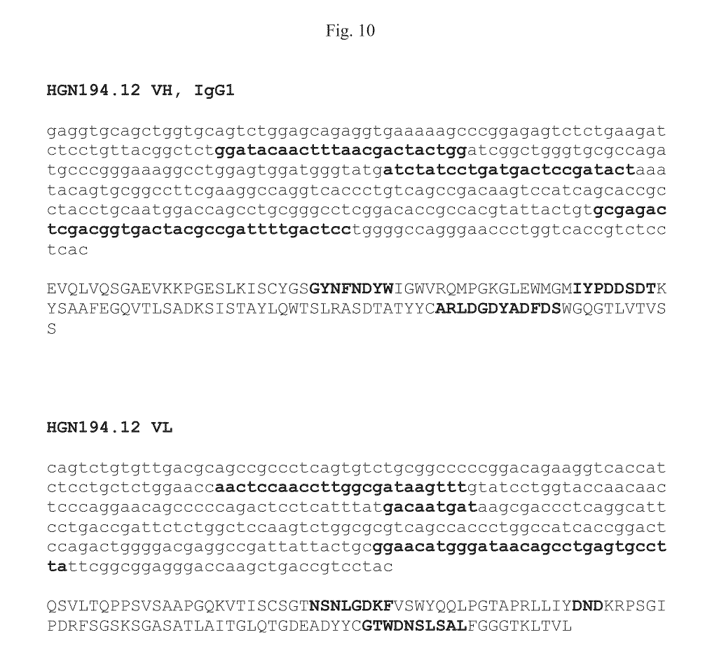

The epitopes recognized by the 55 remaining mAbs were mapped using three experimental approaches: i) cross-competition against mAbs of known specificity or soluble CD4; ii) binding to gp120 mutants or gp41 constructs representing different conformational intermediates; iii) Pepscan analysis. The results of the analysis performed on the gp120-specific and gp41-specific mAbs are summarized in Tables 9 and 10 and depicted in FIG. 7.

To define the epitopes to which the anti-HIV-1 human mAbs bind two different approaches were used: scanning of peptide libraries (Pepscan analysis), cross-competition assays and sCD4 binding inhibition.

TABLE 9
Epitope mapping of gp120-specific mAbs

| | | | | | | | Binding to: | | | | Neutralization | |
|---|---|---|---|---|---|---|---|---|---|---|---|---|
| | Cross-competition with: | | | | | | YU2 | YU2 | YU2 | | | TZM- |
| mAbs | HR10 | HGA9 | HJ16 | b12 | 2G12 | sCD4 | wt | I420R | D368R | Specificity | HOS | bl |
| HGF9 | + | +/− | − | − | − | − | − | − | − | V3 | 4/20 | 6/46 |
| HGT4 | + | +/− | − | − | − | − | − | − | − | V3 | 2/20 | 2/36 |
| HGA13 | + | +/− | − | − | − | − | + | + | + | V3 | 2/3 | nd |
| HGA49 | + | +/− | − | − | − | − | + | + | + | V3 | 5/20 | 8/46 |
| HGA9 | + | + | − | − | − | − | + | + | + | V3 | 7/20 | 11/46 |
| HGD129 | x | + | − | − | − | − | + | + | + | V3 | 3/20 | 7/46 |
| HGD65 | x | + | x | x | x | − | + | + | + | V3 | 6/20 | 7/46 |
| HGI95 | + | + | − | − | − | − | + | + | + | V3 | 9/20 | 8/46 |
| HGN194 | + | + | − | − | − | − | + | + | + | V3 | 11/20 | 19/92 |
| HGP21 | x | + | x | x | x | − | + | + | + | V3 | 4/20 | 5/46 |
| HGP51 | x | + | x | x | x | − | + | + | + | V3 | 4/20 | 5/46 |
| HGW48 | − | + | − | − | − | − | + | + | + | V3 | 6/20 | nd |
| HR10 | + | + | − | − | − | − | + | + | + | V3 | 8/20 | 9/46 |
| HZ74 | + | + | − | − | − | − | + | + | + | V3 | 6/20 | 12/46 |
| HGP27 | − | + | − | − | − | − | + | +/− | + | V3 | 1/3 | nd |
| HX44 | − | − | − | x | x | − | + | − | + | CD4i | 3/20 | 2/46 |
| HGP 105 | − | − | − | x | x | − | + | − | + | CD4i | 1/20 | 1/46 |
| HGW7 | − | − | − | x | x | − | + | − | + | CD4i | 1/20 | nd |
| HGY38 | − | − | − | x | x | − | + | − | + | CD4i | 2/3 | nd |
| HGD14 | x | − | x | + | − | +/− | + | − | − | CD4bs~ | 2/20 | 5/46 |
| HR15 | − | − | − | x | − | +/− | − | − | − | CD4bs~ | 1/3 | nd |
| HGF12 | − | − | − | − | − | +/− | + | + | + | CD4bs~ | 0/3 | 0/46 |
| HGI46 | x | − | x | x | − | +/− | + | +/− | + | CD4bs~ | 1/20 | 5/46 |
| HGI75 | − | − | +/− | x | − | +/− | + | +/− | − | CD4bs~ | 3/20 | 2/46 |
| HGP172 | − | − | +/− | + | − | +/− | + | +/− | − | CD4bs~ | 4/20 | 6/46 |
| HGP134 | − | − | +/− | x | − | + | +/− | − | − | CD4bs | 6/20 | 4/46 |
| HGI111 | − | − | +/− | + | + | + | +/− | − | − | CD4bs | 7/20 | 4/46 |
| HGP31 | − | − | x | +/− | + | + | +/− | − | − | CD4bs | 2/3 | 5/45 |
| HGP61 | − | − | +/− | x | + | + | +/− | − | − | CD4bs | 3/20 | 8/46 |
| HGP82 | − | − | + | + | − | + | + | +/− | − | CD4bs | 7/20 | 8/46 |
| HGS2 | − | − | − | + | − | + | + | +/− | +/− | CD4bs | 7/20 | 4/46 |
| HGW26 | − | − | − | + | − | + | + | +/− | +/− | CD4bs | 3/20 | nd |
| HGZ1 | − | − | +/− | + | − | + | + | +/− | +/− | CD4bs | 8/20 | 10/46 |
| HJ16 | x | − | + | x | − | + | +/− | +/− | + | CD4bs | 10/20 | 33/92 |
| HGP68 | − | − | − | x | − | − | + | +/− | + | V2 | 6/20 | 4/46 |
| HR12 | − | − | − | x | − | − | − | − | − | ? | 0/20 | 2/46 |
| HP12 | x | − | − | x | − | − | − | − | − | ? | 0/20 | 0/46 |
| b12 | − | − | +/− | + | − | + | + | + | − | CD4bs | 11/20 | 43/92 |

Cross competition with biotinylated mAbs of known specificity or sCD4: +, 90-100% inhibition, +/−, 50-90% inhibition, −, <50% inhibition.
The x indicates that it was not feasible to evaluate the competition due to the lack of binding to the immobilized gp120 protein.
Binding by ELISA to YU2 gp120 wt and mutants in the CD4i and the CD4bs.
Specificity assignment (CD4bs~ indicates partial inhibition of sCD4 binding).
Shown is also the number of isolates neutralized in the HOS-based and TZM-bl based assays.

A first group of mAbs was assigned to the V3 loop based on cross-competition with two V3 loop specific mAbs (HR10 and HGA9). This group comprised 8 mAbs, which were already mapped to the V3 loop using the Pepscan analysis approach, and 7 additional mAbs that were not analyzed by peptide scanning (i.e. HGF9, HGT4, HGP21, HGP51, HGW48, HGD129 and HGP27). Four mAbs (HX44, HGP105, HGW7, HGY38) reacted with wt and CD4bs YU2 mutants but failed to bind the CD41 mutant, and therefore were assigned to the CD41 cluster. Notably, one mAb (HGP68) was mapped by Pepscan analysis to a novel epitope in the V2 loop (TVYALFYRLDIVP (SEQ ID NO: 56) and neutralized 4 Tier-1 isolates (FIG. 3). Fifteen other mAbs were assigned tentatively or conclusively to the CD4bs based on their capacity to inhibit gp120-sCD4 binding. Most of these mAbs competed other CD4bs-specific mAbs, such as b12 and HJ16, while in some cases this assay could not be performed due to lack of epitope expression on the gp120 proteins used. Furthermore, this group of mAbs showed variable reactivities with the YU2 mutants. While most of them did not bind the D368R mutant similar to b12, others including HJ16, bound this mutant avidly. In addition, most of these new CD4bs-specific mAbs also showed decreased binding to the CD4i mutant, suggesting that they may span a broader, as yet undescribed region that includes elements of the CD4bs and CD4i sites. Finally, for 2 mAbs these analyses did not provide any relevant information.

Twenty-one mAbs bound to gp41 by targeting primarily the immunodominant C-C region, the HR-1 and the region recognized by 5F3 mAb (Buchacher A, et al. (1994) AIDS Res Hum Retroviruses 10: 359-369). (Table 10).

TABLE 10
Epitope mapping gp41-specific mAbs

| | Cross-competition with: | | | | | Binding to: | | | | | Neutralization | |
|---|---|---|---|---|---|---|---|---|---|---|---|---|
| mAbs | 2F5 | 4E10 | 3D6 | 5F3 | HK20 | HR1 | 5HB | HR2 | Epitope | Specificity | HOS | TZM-bl |
| HGN158 | − | − | − | − | − | − | + | − | | 5HB | 0/3 | nd |
| HGN36 | − | − | − | − | − | − | + | − | | 5HB | 0/20 | 0/46 |
| HGW34 | − | − | − | − | + | +/− | + | − | | 5HB | 0/3 | nd |
| HGW46 | − | − | − | − | − | − | + | − | | 5HB | 0/20 | 0/46 |
| HGK129 | − | − | + | − | − | − | − | + | LLGIWGCSGKLIC | C-C | 0/3 | nd |
| HGN146 | − | − | + | − | − | − | − | + | LLGIWGCSGKLIC | C-C | 2/20 | nd |
| HGN35 | − | − | + | − | − | − | − | + | SGKLIC | C-C | 0/3 | nd |
| HGW17 | − | − | + | − | − | − | − | − | | C-C | 0/20 | nd |
| HGY25 | − | − | + | − | − | − | − | − | | C-C | 0/20 | nd |
| HGP40 | − | − | − | − | − | − | − | − | | gp41 only | 0/20 | 0/46 |
| HGP48 | − | − | − | − | − | − | − | − | | gp41 only | 0/3 | nd |
| HGY50 | − | − | − | − | − | − | − | − | | gp41 only | 0/20 | 0/46 |
| HK20 | − | − | − | − | + | + | + | − | QQHLLQLTVWGIKQL | HR-1 | 17/20 | 3/92 |
| HGB33 | − | − | − | − | + | | + | − | | HR1/5HB | 2/20 | 0/46 |
| HGW63 | − | − | − | + | + | + | + | − | | HR1/5HB | 0/20 | nd |
| HGD161 | − | − | − | + | + | + | + | − | | HR-1/FP | 0/20 | 0/46 |
| HGP16 | − | − | − | + | + | + | + | +/− | | HR-1/FP | 1/20 | 1/46 |
| HGW23 | − | − | − | +/− | +/− | + | + | − | | HR-1/FP | 3/20 | nd |
| HGN91 | − | − | − | +/− | +/− | + | + | − | | HR-1/FP | 0/3 | nd |
| HGH8 | − | − | − | +/− | +/− | − | + | − | | HR-1/FP | 0/20 | 0/46 |
| HGF24 | − | − | − | − | +/− | − | + | + | TNLIYTLIEESQN | HR-2 | 2/20 | 4/46 |
| 3D6 | − | − | + | − | − | − | − | +/− | GCSGKLICTTAVPW | C-C | nd | nd |
| 5F3 | − | − | − | + | − | + | + | | STMGAASITLTAQARQ | FP | nd | nd |
| 2F5 | +/− | +/− | − | − | − | − | − | +/− | DKW | MPER | 10/20 | 35/90 |
| 4E10 | + | + | − | − | − | − | − | +/− | WFDI | MPER | 20/20 | 89/90 |

Cross competition with biotinylated mAbs of known specificity: +,90-100% inhibition, +/−, 50-90% inhibition, −, <50% inhibition.
Binding to different gp41 constructs by ELISA.
Minimal linear epitopes using the Pepscan analysis and specificity assignment.
Shown is also the number of isolates neutralized in the HOS-based and TZM-bl based assays.

MAbs HGK129, HGN146 and HGN35 were assigned to the C-C loop since they competed with 3D6, reacted with an HR2 construct and recognized synthetic peptides within this region. MAbs HGW17 and HGY25 were provisionally assigned to the CC region since they competed with 3D6. These data are consistent with the immunodominance of the C-C region and with the notion that the antibody response against this region overlaps with the 3D6 epitope. Several mAbs competed with 5F3 and HK20, and bound both the HR-1 and 5HB constructs, suggesting that they may bind between the fusion peptide and the HR-1 region. HGB33 was provisionally assigned to the HR-1 region according to competition with HK20 and binding to the 5HB construct. Other mAbs bound specifically the 5HB construct but did not compete with any of the mAbs tested, thus indicating that the complete HR-1 coiled coil region exposed in 5HB harbors antibody epitopes available for B cell recognition in the gp41 pre-hairpin conformation. The concomitant binding to recombinant gp41 suggests that the latter may partially resemble the gp41 pre-fusion state. Of note, three gp41 binding mAbs (HGP40, HGP48 and HGY50) neither competed with any of the mAbs tested, nor bound to any constructs representing pre-hairpin conformations or native gp140 (FIG. 1), indicating that they recognize as yet uncharacterized regions that are only available in the recombinant gp41 protein. Interestingly, mAb HGF24 was assigned by Pepscan analysis to an epitope in the C-terminal region of HR-2 (TNLIYTLIEESQN (SEQ ID NO: 57)), proximal to the 2F5 epitope, and neutralized 4 Tier-2 isolates (data not shown). This mAb competed with HK20 for gp41 binding, in spite of their distinct cognate specificities (HR-2 and HR-1, respectively). This finding would be consistent with the proximity of HR-1 and HR2 in the six-helix bundle structure. Finally, although the gp41 protein used in the primary screening includes the 2F5 and 4E10 epitopes, no MPER specific mAbs were isolated, reinforcing the idea that this portion of gp41 is poorly immunogenic in humans. In conclusion, regardless of their limited breadth of neutralization, this extended panel of human mAbs may represent a useful tool for understanding the molecular basis of Env recognition in humans.

### Examples of Antibodies

HJ16

Specificity: anti CD4 binding site. Preferential neutralization of Tier 2 viruses (more resistant strains than Tier 1 viruses). Epitope may be discontinuous but not necessarily a conformational epitope (may form a beta loop). In contrast, b12, a similar antibody, has an epitope that is composed of loops very distant in three-dimensional structure. Thus, epitope recognized by HJ16 may be easier to get for vaccine purposes.

HK20

Specificity: heptad repeat (HR) 1 trimer. Broad neutralization profile but epitope poorly accessible. Epitope exposed at the pre-fusion stage. Fab fragment and scFv have a higher activity compared to intact mAb. Germline usage is VH1-69. The residues of the epitope in the HXB2 gp41 sequence (which is a reference sequence) that are recognized by antibody HK20 comprise: His 564, Leu 565, Gln 567, Leu 568, Thr 569, Val 570, Trp 571, Ile 573, Lys 574, Gln 575, Gln 577, Ala 578, Ala 582 and His 643. The HK20 contact residues of the heavy chain are (Kabat numbering): Ile 53, Phe 54, Asp 55, Ile 56, Asn 58, Ser 96, Tyr 97, Ser 98, Ser 99, Ser 100a, Pro 100b and Tyr 100c. The light chain interaction residues are: Asp 93 and Leu 94.

HGN194

Specificity: V3 loop. Pepscan analysis identified the epitope which is very conserved in the tip of V3 of gp120. It includes 4 residues involved in co-receptor (CCR5 or CXCR4) binding. Also HGN194 has accessibility problems (Fab fragment and single chain antibody may work better). Neutralizes most Tier 1 isolates (they are more sensitive isolates since the epitope is more accessible). In vivo administration in macaques show efficacy achieving complete protection.

All patents and publications referred to herein are expressly incorporated by reference in their entirety.

It should be noted that there are alternative ways of implementing the present invention and that various modifications can be made without departing from the scope and spirit of the invention. Accordingly, the present embodiments are to be considered as illustrative and not restrictive, and the invention is not to be limited to the details given herein, but may be modified within the scope and equivalents of the appended claims.

1. An antibody, or an antigen binding fragment thereof, comprising at least one complementarity determining region (CDR) sequence having at least 95% sequence identity to any one of SEQ ID NOs: 17-22, 1-6, or 33-38, wherein the antibody or antigen binding fragment neutralizes HIV-1 infection.

2. The antibody of claim 1, or an antigen binding fragment thereof, comprising a heavy chain CDR1 selected from the group consisting of SEQ ID NOs: 17, 1 and 33; a heavy chain CDR2 selected from the group consisting of SEQ ID NOs: 18, 2 and 34; and a heavy chain CDR3 selected from the group consisting of SEQ ID NOs: 19, 3 and 35, wherein the antibody or antigen binding fragment neutralizes HIV-1 infection.

3. The antibody of claim 1, or an antigen binding fragment thereof, comprising a light chain CDR1 selected from the group consisting of SEQ ID NOs: 20, 4 and 36; a light chain CDR2 selected from the group consisting of SEQ ID NOs: 21, 5 and 37; and a light chain CDR3 selected from the group consisting of SEQ ID NOs: 22, 6 and 38, wherein the antibody or antigen binding fragment neutralizes HIV-1 infection.

4. The antibody of claim 1, or an antigen binding fragment thereof, comprising a heavy chain comprising SEQ ID NO: 17 for CDRH1, SEQ ID NO; 18 for CDRH2, SEQ ID NO: 19 for CDRH3; or SEQ ID NO: 1 for CDRH1, SEQ ID NO: 2 for CDRH2, SEQ ID NO: 3 for CDRH3; or SEQ ID NO; 33 for CDRH1, SEQ ID NO: 34 for CDRH2, SEQ ID NO: 35 for CDRH3.

5. The antibody of claim 1, or an antigen binding fragment thereof, comprising a light chain comprising SEQ ID NO: 20 for CDRL1, SEQ ID NO: 21 for CDRL2, SEQ ID NO: 22 for CDRL3; or SEQ ID NO: 4 for CDRL1, SEQ ID NO: 5 for CDRL2, SEQ ID NO: 6 for CDRL3; or SEQ ID NO: 36 for CDRL1, SEQ ID NO: 37 for CDRL2, SEQ ID NO: 38 for CDRL3.

6. The antibody of claim 1, or an antigen binding fragment thereof, comprising a heavy chain variable region having at least 80% sequence identity to any one of SEQ ID NOs: 29, 13 or 45.

7. The antibody of claim 1, or an antigen binding fragment thereof, comprising a light chain variable region having at least 80% sequence identity to any one of SEQ ID NOs: 30, 14 or 46.

8. The antibody of claim 1, or an antigen binding fragment thereof, wherein the antibody comprises a heavy chain variable region comprising the amino acid sequence of any one of SEQ ID NO: 29, SEQ ID NO: 13 or SEQ ID NO: 45; and wherein the antibody comprises a light chain variable region comprising the amino acid sequence of SEQ ID NO: 30, SEQ ID NO: 14, or SEQ ID NO: 46.

9. An antibody, or an antigen binding fragment thereof, wherein the antibody comprises a heavy chain variable region comprising the amino acid sequence of SEQ ID NO: 29 and a light chain variable region comprising the amino acid sequence of SEQ ID NO: 30; or a heavy chain variable region comprising the amino acid sequence of SEQ ID NO: 13 and a light chain variable region comprising the amino acid sequence of SEQ ID NO: 14; or a heavy chain variable region comprising the amino acid sequence of SEQ ID NO: 45 and a light chain variable region comprising the amino acid sequence of SEQ ID NO: 46, wherein the antibody or antigen binding fragment neutralizes HIV-1 infection.

10. The antibody of claim 1, or an antigen binding fragment thereof, wherein the antibody is HK20, HJ16 or HGN194.

11. The antibody of claim 1, or an antigen binding fragment thereof, wherein the antibody is a monoclonal antibody, a purified antibody, an isolated antibody, a single chain antibody, Fab, Fab′, F(ab′)2, Fv or scFv.

12. The antibody of claim 1, or an antigen binding fragment thereof, that specifically binds an epitope in a gp41 protein, wherein the epitope comprises His 564, Leu 565, Gln 567, Leu 568, Thr 569, Val 570, Trp 571, Ile 573, Lys 574, Gln 575, Gln 577, Ala 578, Ala 582 and His 643, wherein the antibody or antigen binding fragment thereof neutralizes HIV-1 infection and wherein the antibody is not D5.

13. An isolated antibody, or an antigen binding fragment thereof, that (i) binds to the same epitope as the antibody or antibody fragment of claim 1, wherein the antibody or antigen binding fragment thereof neutralizes HIV-1 infection, and wherein the antibody is not D5; or (ii) cross competes with an antibody or antibody fragment of claim 1, wherein the antibody or antigen binding fragment thereof neutralizes HIV-1 infection, and wherein the antibody is not D5.

14. The antibody of claim 1, or an antigen binding fragment thereof, that neutralizes infection of HIV-1 Tier 1 isolates.

15. The antibody of claim 1, or an antigen binding fragment thereof, that neutralizes infection of HIV-1 Tier 2 isolates.

16. The antibody of claim 1, or an antigen binding fragment thereof, that neutralizes infection of HIV-1 Tier 1 and Tier 2 isolates.

17. The antibody of claim 1, or an antigen binding fragment thereof, for treatment of HIV-1 infection.

18. A nucleic acid molecule comprising a polynucleotide encoding the antibody, or an antigen binding fragment thereof, of claim 1.

19. The nucleic acid molecule of claim 18, wherein the polynucleotide sequence is at least 75% identical to the nucleic acid sequence of any one of SEQ ID NOs: 23-28, 31, 32, 7-12, 15, 16, 39-44, 47 or 48.

20. A cell expressing the antibody of claim 1, or an antigen binding fragment thereof.

21. A pharmaceutical composition comprising the antibody of claim 1, or an antigen binding fragment thereof; a nucleic acid molecule comprising a polynucleotide encoding said antibody, or an antigen binding fragment thereof; a vector comprising said polynucleotide; a cell expressing said vector; or an isolated or purified immunogenic polypeptide comprising an epitope that binds to said antibody, or an antigen binding fragment thereof, and a pharmaceutically acceptable diluent or carrier.

22. A pharmaceutical composition comprising a first antibody or an antigen binding fragment thereof, and a second antibody, or an antigen binding fragment thereof, wherein the first antibody is the antibody of claim 1, and the second antibody neutralizes HIV-1 infection.

23. Use of the antibody of claim 1, or an antigen binding fragment thereof; a nucleic acid molecule comprising a polynucleotide encoding said antibody, or an antigen binding fragment thereof; a vector comprising said polynucleotide; a cell expressing said vector; an isolated or purified immunogenic polypeptide comprising an epitope that binds to said antibody, or an antigen binding fragment thereof; or a pharmaceutical composition comprising said antibody, antibody fragment, nucleic acid molecule, vector, cell or isolated or purified immunogenic polypeptide (i) in the manufacture of a medicament for the treatment of HIV-1 infection, (ii) in a vaccine, (iii) in diagnosis of HIV-1 infection, or (iv) for monitoring the quality of anti-HIV-1 vaccines by checking that the antigen of said vaccine contains the specific epitope in the correct conformation.

24. A method of reducing HIV-1 infection, or lowering the risk of HIV-1 infection, comprising: administering to a subject in need thereof, a prophylactically or therapeutically effective amount of the antibody of claim 1, or an antigen binding fragment thereof.

25. An isolated or purified immunogenic polypeptide comprising an epitope that specifically binds to the antibody of claim 1, or an antigen binding fragment thereof, for use (i) in therapy, (ii) in the manufacture of a medicament for treating HIV-1 infection, (iii) as a vaccine, or (iv) in screening for ligands able to neutralize HIV-1 infection.
